(19) United States
(12) Patent Application Publication
(10) Pub. No.: US 2017/0307615 A1
(43) Pub. Date: Oct. 26, 2017

(54) Immunohistochemistry Quality Management Program Using Cultured Cell Lines for Tissue Microarray (TMA) Blocks

(71) Applicant: Geisinger Health System, Danville, PA (US)

(72) Inventor: Fan Lin, Lewisburg, PA (US)

(73) Assignee: Geisinger Health System, Danville, PA (US)

(21) Appl. No.: 15/512,979

(22) PCT Filed: Sep. 24, 2015
(86) PCT No.: PCT/US2015/051890

§ 371 Date: Mar. 21, 2017

Related U.S. Application Data
(60) Provisional application No. 62/054,483, filed on Sep. 24, 2014.

(51) Int. Cl. G01N 33/574; G01N 1/36

(52) U.S. Cl. CPC G01N 33/574; G01N 1/36; G01N 2001/368

(57) ABSTRACT

Provided are methods for producing a Tissue Microarray (TMA) Block from a mixture of cultured cell lines for use as an immunohistochemical control block in diagnosis and/or prognosis of one or more particular cancers. Provided are Tissue Microarray (TMA) Blocks for use as an immunohistochemical control block in diagnosing of one or more particular cancers. Provided are Universal Tissue Microarray Blocks for use as an immunohistochemical control block in differential diagnosing of one or more of carcinoma, melanoma, germ cell tumor, sarcoma, and lymphoma.

### CROSS-REFERENCE TO RELATED APPLICATION

This application claims priority to U.S. Provisional Patent Application Ser. No. 62/054,483, filed on Sep. 24, 2014, which is incorporated herein by reference in its entirety for all purposes.

### FIELD OF THE INVENTION

The invention relates to methods of constructing TMA blocks from cultured cell lines with a mixture of cell lines in the desired ratios for use in an IHC quality management program.

### BACKGROUND OF THE INVENTION

Immunohistochemistry (IHC) has become an important ancillary study technique in diagnostic surgical pathology and cytopathology. The clinical utility of IHC encompasses tumor diagnosis, differential diagnosis, and prognostic/predictive markers.

An IHC assay is a high-complexity test which includes many complicated working steps in the pre-analytic, analytic, and post-analytic phases. Any errors occurring in any of these steps may cause unreproducible and unreliable results. A total IHC quality management program (quality assurance, quality control and quality improvement) needs to be implemented to minimize potential errors in a clinical IHC lab. In analytic and post-analytic phases, tissue microarray (TMA) blocks containing various numbers of tumors and/or normal tissues have proven to be extremely valuable for 1) external positive and negative control tissues; 2) new antibody testing and optimization; 3) antibody validation; and 4) continuous quality monitoring of commonly used antibodies.

The vast majority of IHC Labs in the United States (US) use automated IHC stainers to perform IHC stains. Both vendors (such as Ventana) and some quality control organizations, such as College of American Pathologists (CAP), require one external positive IHC control section/slide for each patient IHC stain slide. Because of this, nearly the same number of external positive control sections/slides is needed for the same number of patient slides in a clinical IHC lab. As such, millions of IHC external positive control sections/slides are used in the US IHC labs every year.

Currently, in all IHC labs in the US, either tumor tissue blocks or normal tissue blocks are used as external positive control slides, depending upon the antibodies being ordered. The positive control blocks can be constructed by each IHC lab or ordered from a commercial company. The cost for each positive control slide varies for a given antibody. Instead of purchasing from a commercial lab, many IHC labs build the majority of their positive control blocks, with the exception of purchasing positive control slides for rare antibodies. But IHC tech time is expensive as well, and this also involves the consumption of valuable tumor blocks from the pathology archives, which are important for future molecular testing, clinical trials, and research. Thus, there remains a need in the art for improved methods and supplies for IHC quality management.

### SUMMARY OF THE INVENTION

The present invention pertains to TMA blocks comprising a mixture of cultured cell lines, as well as methods of their production and use.

In one aspect, the method comprises identifying a plurality of positive biomarkers useful in diagnosis of and/or prognosis of one or more particular cancers; optionally measuring the expression of said positive biomarkers in two or more cell lines; selecting: (i) one or more high-positive cell lines that each have a high level of expression of one or more of the plurality of positive biomarkers such that the high-positive cell lines collectively provide high level expression of all of said positive biomarkers, and one or both of: (ii) one or more low-positive cell lines that each have a low level of expression of one or more of said positive biomarkers, and (iii) one or more null-positive cell lines that each have no expression of one or more of said positive biomarkers, wherein a single selected cell line may be from both groups i) and ii); i) and iii); ii) and iii); or i), ii), and iii) for different positive biomarkers; mixing cells of said selected cell lines; and producing a TMA block from said mixed cells.

In certain embodiments of the invention, the low-positive cell lines collectively provide low level expression of the majority of said plurality of positive biomarkers. In other embodiments of the invention, the low-positive cell lines collectively provide low level expression of all of said plurality of positive biomarkers. In further embodiments of the invention, the null-positive cell lines collectively provide no expression of the majority of said positive biomarkers. In yet other embodiments of the invention, the null-positive cell lines collectively provide no expression of all of said positive biomarkers.

In some embodiments of the invention, the method further comprises identifying one or more negative biomarkers useful in diagnosing one or more particular cancers; optionally measuring the expression of said negative biomarkers; and selecting one or more high-negative cell lines that each have a low level or no expression of positive biomarkers and a high level of expression of one or more of the negative biomarkers; wherein a single selected cell line may be both a high-negative cell line and a null-positive cell line, or a single selected cell line may be both a high-negative cell line and a low-positive cell line.

In some embodiments of the invention, the high-negative cell lines collectively provide high level expression of the majority of said negative biomarkers. In other embodiments of the invention, the high-negative cell lines collectively provide high level expression of all of said negative biomarkers.

In certain embodiments of the invention, the method further comprises determining the combination ratio of selected cell lines to create a TMA block. In some embodiments of the invention, the ratio produces a TMA block with between about 20% to about 80% of cells having a high level of expression of each of said positive biomarkers. In other embodiments of the invention, the ratio produces a TMA block with between about 20% to about 80% of cells having a low level of expression of one or more of said positive biomarkers. In further embodiments of the invention, the ratio produces a TMA block with between about 20% to about 80% of cells having no expression of one or more of said positive biomarkers. In yet other embodiments of the invention, the ratio produces a TMA block with between about 20% to about 80% of cells having a high level of expression of each of one or more of said negative biomarkers.

The present invention also pertains to TMA blocks useful in diagnosing one or more particular cancers. In certain embodiments of the invention, the TMA block is a Melanoma Control Block comprising G361 cells, CRL-1585 cells, and CRL-5895 cells, wherein total cells used are about 20-60% of said G361 cells, about 20-60% of said CRL-1585 cells, and about 5-40% of said CRL-5895 cells. In other embodiments of the invention, the Melanoma Control Block comprises about 30-50% of said G361 cells, about 30-50% of said CRL-1585 cells, and about 10-30% of said CRL-5895 cells. In particular embodiments of the invention, the Melanoma Control Block comprises about 40% of said G361 cells, about 40% of said CRL-1585 cells, and about 20% of said CRL-5895 cells.

In other embodiments of the invention, the TMA block is a Breast Cancer Control Block comprising HTB133, CRL-2330, and CCL-253 cells, wherein total cells used are about 20-60% of said HTB-133 cells, about 20-60% of said CCL-253 cells, and about 5-40% of said CRL-2330 cells. In certain embodiments of the invention, the Breast Cancer Control Block comprises about 30-50% of said HTB-133 cells, about 30-50% of said CCL-253 cells, and about 10-30% of said CRL-2330 cells. In particular embodiments of the invention, the Breast Cancer Control Block comprises about 40% of said HTB-133 cells, about 40% of said CCL-253 cells, and about 20% of said CRL-2330 cells.

In further embodiments, the TMA block is a Lymphoma/Hematopoietic Tumor Control Block comprising CRL-1582-Molt4, CCL-86-BLM, and CRL-5895, wherein total cells used are about 20-60% of said CRL-1582-Molt4 cells, about 20-60% of said CCL-86-BLM cells, and about 5-40% of said CRL5895 cells. In certain embodiments, the Lymphoma/Hematopoietic Tumor Control Block comprises about 30-50% of said CRL-1582-Molt4 cells, about 30-50% of said CCL-86-BLM cells, and about 10-30% of said CRL5895 cells. In particular embodiments, the Lymphoma/Hematopoietic Tumor Control Block comprises about 40% of said CRL-1582-Molt4 cells, about 40% of said CCL-86-BLM cells, and about 20% of said CRL5895 cells.

In yet other embodiments of the invention, the TMA block is a Germ Cell Tumor Control Block comprising CRL-2073, HTB-36, HepG2, and CRL-1585, wherein total cells used are about 5-45% of said CRL-2073 cells, about 5-45% of said HTB-36 cells, about 5-45% of said HepG2 cells, and about 5-45% of said 09-c-CRL-1585 cells. In certain embodiments of the invention, the Germ Cell Tumor Control Block comprises about 15-35% of said CRL-2073 cells, about 15-35% of said HTB-36 cells, about 15-35% of said HepG2 cells, and about 15-35% of said 09-c-CRL-1585 cells. In particular embodiments of the invention, the Germ Cell Tumor Control Block comprises about 25% of said CRL-2073 cells, about 25% of said HTB-36 cells, about 25% of said HepG2 cells, and about 25% of said 09-c-CRL-1585 cells.

In other embodiments of the invention, the TMA block is a Malignant Small Round Cell/Blue Cell Tumor Control Block comprising CCL-136, HTB166, TT, CRL-5946, CRL-1582-Molt4, and CCL-86-BLM, wherein total cells used are about 5-40% of said CCL-136 cells, about 5-40% of said HTB166 cells, about 5-40% of said TT cells, about 5-40% of said CRL-5946, about 5-30% of said CRL-1582-Molt4, and about 5-30% of said CCL-86-BLM cells. In certain embodiments of the invention, the Malignant Small Round Cell/Blue Cell Tumor Control Block comprises about 10-30% of said CCL-136 cells, about 10-30% of said HTB166 cells, about 10-30% of said TT cells, about 10-30% of said CRL-5946, about 5-20% of said CRL-1582-Molt4, and about 5-20% of said CCL-86-BLM cells. In particular embodiments of the invention, the Malignant Small Round Cell/Blue Cell Tumor Control Block comprises about 20% of said CCL-136 cells, about 20% of said HTB166 cells, about 20% of said TT cells, about 20% of said CRL-5946, about 10% of said CRL-1582-Molt4, and about 10% of said CCL-86-BLM cells.

In certain embodiments of the invention, the TMA block is a Sarcoma/Spindle Cell Neoplasm Control Block comprising HTB166, CCL-136, CRL-2279, CRL-1585, and CRL-1550, wherein total cells used are about 5-40% of said HTB166 cells, about 5-40% of said CRL-136 cells, about 5-40% of said CRL-2279 cells, about 5-40% of said CRL-1585 cells, and about 5-40% of said CRL-1550 cells. In other embodiments of the invention, the Sarcoma/Spindle Cell Neoplasm Control Block comprises about 10-30% of said HTB166 cells, about 10-30% of said CRL-136 cells, about 10-30% of said CRL-2279 cells, about 10-30% of said CRL-1585 cells, and about 10-30% of said CRL-1550 cells. In particular embodiments of the invention, the Sarcoma/Spindle Cell Neoplasm Control Block comprises about 20% of said HTB166 cells, about 20% of said CRL-136 cells, about 20% of said CRL-2279 cells, about 20% of said CRL-1585 cells, and about 20% of said CRL-1550 cells.

In further embodiments of the invention, the TMA block is a Tumor of Unknown Primary Control Block comprising HTB133, NCI-H508, TT, Pan 3.27, CRL-1932, CRL-2279, CRL-1550, and CRL-5946, wherein total cells used are about 5-25% of said HTB133 cells, about 5-25% of said NCI-H508 cells, about 10-30% of said TT cells, about 5-20% of said Pan3.27 cells, about 5-20% of said CRL-1932 cells, about 5-20% of said CRL-2279 cells, about 5-20% of said CRL-1550 cells, and about 5-20% of said CRL-5946 cells. In certain embodiments of the invention, the Tumor of Unknown Primary Control Block comprises about 10-20% of said HTB133 cells, about 10-20% of said NCI-H508 cells, about 15-25% of said TT cells, about 5-15% of said Pan3.27 cells, about 5-15% of said CRL-1932 cells, about 5-15% of said CRL-2279 cells, about 5-15% of said CRL-1550 cells, and about 5-15% of said CRL-5946 cells. In particular embodiments of the invention, the Tumor of Unknown Primary Control Block comprises about 15% of said HTB133 cells, about 15% of said NCI-H508 cells, about 20% of said TT cells, about 10% of said Pan3.27 cells, about 10% of said CRL-1932 cells, about 10% of said CRL-2279 cells, about 10% of said CRL-1550 cells, and about 10% of said CRL-5946 cells.

In certain embodiments of the invention, the TMA block is a Melanoma Control Block comprising cells of a plurality of cultured cell lines, wherein said cultured cell lines collectively provide high-level expression and no expression of a plurality, one or more, two or more, three or more, four or more, five or more, six or more, seven or more, eight or more, or all of positive biomarkers S100, HMB45, MiTF, Mart-1, SOX10, SOX2, MUM1, S100A6, and Vimentin; low-level expression of a plurality, one or more, two or more, three or more, four or more, five or more, six or more, seven or more, eight or more, or all of biomarkers S100, HMB45, MiTF, Mart-1, SOX10, SOX2, MUM1, S100A6, and Vimentin; and high-level expression and no expression of a plurality, one or more, or all of negative biomarkers Cytokeratin and Cytokeratin 7.

In other embodiments of the invention, the TMA block is a Breast Cancer Control Block comprising cells of a plurality of cultured cell lines, wherein said cultured cell lines collectively provide high level expression, low level expression, and no expression of a plurality, one or more, two or more, three or more, four or more, five or more, six or more, seven or more, eight or more, or all of biomarkers HER2, estrogen receptors (ER), progesterone receptors (PR), GATA3, GCDPF15, mammaglobin, TFF1, TFF3, and CK7.

In further embodiments of the invention, the TMA block is a Lymphoma/Hematopoietic Tumor Control Block comprising cells of a plurality of cultured cell lines, wherein said cultured cell lines collectively provide high level expression, low level expression, and no expression of a plurality, one or more, two or more, three or more, four or more, five or more, six or more, seven or more, eight or more, nine or more, ten or more, eleven or more, twelve or more, thirteen or more, fourteen or more, or all of biomarkers CD2, CD3, CD5, CD7, CD10, CD20, CD79a, PAX5, Bcl2, Bcl6, EBV, TdT, CD99, CK, and CK7.

In yet other embodiments of the invention, the TMA block is a Germ Cell Tumor Control Block comprising cells of a plurality of cultured cell lines, wherein said cultured cell lines collectively provide high level expression, low level expression, and no expression of a plurality, one or more, two or more, three or more, four or more, five or more, six or more, seven or more, eight or more, nine or more, ten or more, eleven or more, or all of biomarkers SALL4, OCT4, PLAP, beta-HCG, alpha-fetoprotein, glypican 3, D2-40, CD30, SOX2, Nanog, S100P, and cytokeratin.

In certain embodiments of the invention, the TMA block is a Malignant Small Round Cell/Blue Cell Tumor Control Block comprising cells of a plurality of cultured cell lines, wherein said cultured cell lines collectively provide high level expression, low level expression, and no expression of a plurality, one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, or all of biomarkers desmin, MyoD1, myogenin, smooth muscle actin, CD99, NKX2.2, Fli-1, synaptophysin, chromogranin, CD56, NSE, WT-1, vimentin, TTF1, cytokeratin, CD2, CD3, TdT, CD20, CD79a, and EBV.

In further embodiments of the invention, the TMA block is a Sarcoma/Spindle Cell Neoplasm Control Block comprising cells of a plurality of cultured cell lines, wherein said cultured cell lines collectively provide high level expression, low level expression, and no expression of a plurality, one or more, two or more, three or more, four or more, five or more, six or more, seven or more, eight or more, nine or more, ten or more, eleven or more, twelve or more, thirteen or more, fourteen or more, fifteen or more, or all of biomarkers desmin, MyoD1, myogenin, smooth muscle actin, CD99, NKX2.2, Fli-1, ERG, vimentin, S100, CK5/6, CK903, p63, p40, p16, and cytokeratin.

In yet other embodiments of the invention, the TMA block is a Tumor of Unknown Primary Control Block comprising cells of a plurality of cultured cell lines, wherein said cultured cell lines collectively provide high level expression, low level expression, and no expression of a plurality, one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, twenty-four or more, twenty-five or more, twenty-six or more, twenty-seven or more, twenty-eight or more, twenty-nine or more, thirty or more thirty-one or more, thirty-two or more, thirty-three or more, thirty-four or more, thirty-five or more, thirty-six or more, thirty-seven or more, thirty-eight or more, thirty-nine or more, or all of biomarkers pan cytokeratin, CK7, CK20, CK5/6, CK903, p63, p40, ER, PR, GATA3, GCDFP15, CDX2, SATB2, cadherin-17, CEA, MOC31, BerEP4, beta-cateinin, B72.3, arginase-1, HepPar1, TTF1, calcitonin, chromogranin, synaptophysin, CD56, MUC1, MUC2, MUC5AC, maspin, S100P, PAX2, PAX8, vimentin, P504S, ERG, Fli-1, p16, WT-1, and HPV (in situ).

The present invention also pertains to a Universal Tissue Microarray Block for use as a IHC control block in differential diagnosing. In certain embodiments of the invention, the Universal Tissue Microarray Block is used in differential diagnosing of one or more of carcinoma, melanoma, germ cell tumor, sarcoma, and lymphoma. In further embodiments of the invention, the Universal Tissue Microarray Block comprises a plurality, one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, or all of cell lines G361, CRL-1585, CRL-1582-Molt4, CCL-86-BLM, CRL-2073, HTB-36, HepG2, CCL-136, HTB166, HTB133, CCL-253, NCI-H508, TT, Pan 3.27, CRL-1932, CRL-2279, CRL-1550, and CRL-5946. In further embodiments, the Universal Tissue Microarray Block comprises cell lines wherein each cell line comprises from about 1% to about 25% of the total cells of the Universal Tissue Microarray Block. In other embodiments of the invention, the cell lines of the Universal Tissue Microarray Block collectively provide high level expression, low level expression, and no expression of biomarkers. In further embodiments of the invention, the biomarkers may include a plurality, one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, twenty-four or more, twenty-five or more, twenty-six or more, twenty-seven or more, twenty-eight or more, twenty-nine or more, thirty or more thirty-one or more, thirty-two or more, thirty-three or more, thirty-four or more, thirty-five or more, thirty-six or more, thirty-seven or more, thirty-eight or more, thirty-nine or more, forty or more, forty-one or more, forty-two or more, forty-three or more, forty-four or more, forty-five or more, forty-six or more, forty-seven or more, forty-eight or more, forty-nine or more, fifty or more, fifty-one or more, fifty-two or more, fifty-three or more, fifty-four or more, fifty-five or more, fifty-six or more, fifty-seven or more, fifty-eight or more, fifty-nine or more, sixty or more, sixty-one or more, sixty-two or more, sixty-three or more, sixty-four or more, sixty-five or more, sixty-six or more, sixty-seven or more, sixty-eight or more, or all of S100, HMB45, MiTF, Mart-1, SOX2, SOX10, SALL4, OCT4, PLAP, beta-HCG, AFP, glypican 3, CD30, D2-40, HER2, ER, PR, GATA3, GCDFP15, MGB, CK7, CK20, CK5/6, CK903, p40, p63, CDX2, SATB2, cadherin-17, TTF1, napsin A, calcitonin, chromogranin, synaptophysin, CD56, MUC1, MUC2, MUCSAC, calretinin, MOC31, BerEP4, Arginase-1, HepPar-1, PAX2, PAX8, IMP3, maspin, vimentin, p16, WT-1, HPV, desmin, MyoD1, myogenin, SMA, NKX2.2, Fli-1, ERG, CD2, CD3, CD5, CD10, CD20, CD79a, PAX5, TdT, EBV, Ki-67, and/or p53.

The present invention pertains to the construction of TMA blocks from the cultured cell lines. In some embodiments of the invention, the construction of TMA blocks is done in the following steps: 1) culture the cell lines; 2) build a cell block from each cultured cell line; 3) test the selected biomarkers/antibodies on the constructed cell block; 4) mix the selected cell lines in a desired ratio with the expression of known positive and negative biomarkers; 5) construct tissue microarray (TMA) blocks from the cell blocks containing selected mixed cell lines; and 6) re-test the selected antibodies on the constructed TMA blocks to confirm the expression of the targeted biomarkers. In certain embodiments, the cell lines are obtained from the American Type Culture Collection (ATCC).

### BRIEF DESCRIPTION OF THE DRAWINGS

The foregoing will be apparent from the following more particular description of example embodiments of the invention, as illustrated in the accompanying drawings.

FIG. 1 illustrates the expression of select biomarkers in the breast cancer control block containing the mixture of 3 different cell lines

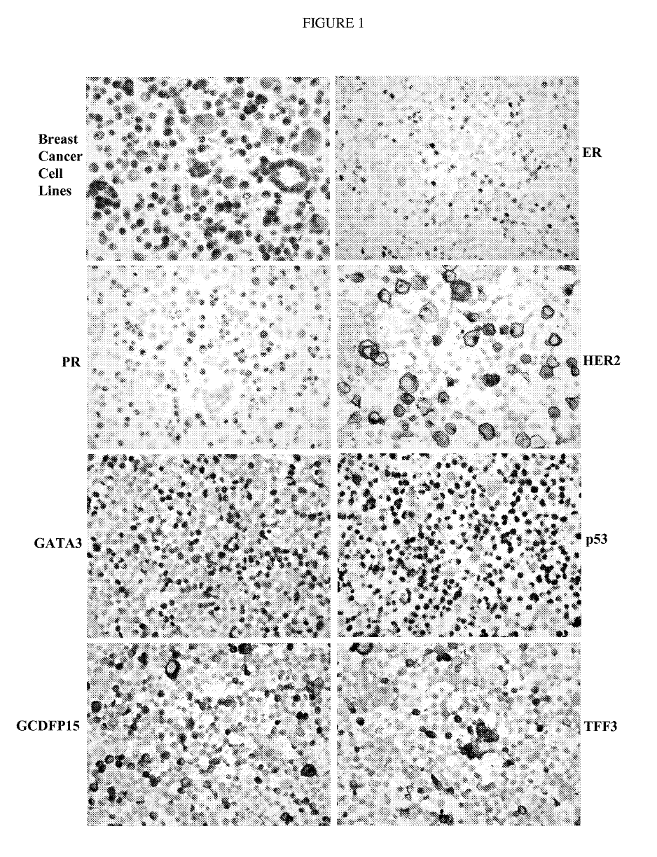

FIG. 2 demonstrates the staining results for the melanoma control block containing the mixture of 3 different cell lines.

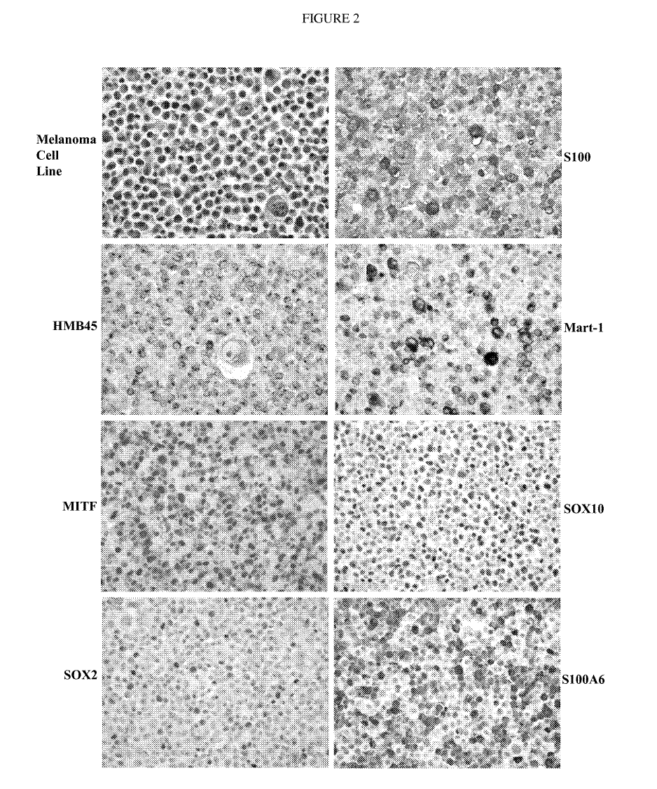

### DETAILED DESCRIPTION OF THE INVENTION

A description of example embodiments of the invention follows.

### Definitions

The present invention may be understood more readily by reference to the following detailed description of preferred embodiments of the invention and the methods included therein. Before the present methods and techniques are disclosed and described, it is to be understood that this invention is not limited to specific analytical or synthetic methods as such may, of course, vary. It is also to be understood that the terminology used herein is for the purpose of describing particular embodiments only and is not intended to be limiting. Unless defined otherwise, all technical and scientific terms used herein have the meaning commonly understood by one of ordinary skill in the art to which this invention belongs.

As used herein, the singular forms “a,” “and,” and “the” include plural reference unless the context clearly dictates otherwise. Thus, for example, reference to “a biomarker” is reference to one or more biomarkers and includes equivalents thereof known to those skilled in the art. Additionally, the term “comprises” is intended to include embodiments where the method, apparatus, composition, etc., consists essentially of and/or consists of the listed steps, components, etc. Similarly, the term “consists essentially of” is intended to include embodiments where the method, apparatus, composition, etc., consists of the listed steps, components, etc.

Numeric ranges recited within the specification and claims are inclusive of the numbers defining the range (the end point numbers) and also are intended to include each integer or any non-integer fraction within the defined range. Further, as used herein, the term “about” refers to a number that differs from the given number by less than 10%. In other embodiments, the term “about” indicates that the number differs from the given number by less than 9%, 8%, 7%, 6%, 5%, 4%, 3%, 2%, or 1%.

The term “antibody,” as used herein, refers to immunoglobulin molecules and immunologically active portions of immunoglobulin molecules. As such, the term antibody can refer to any type, including for example IgG, IgE, IgM, IgD, IgA and IgY, any class, including for example IgG1, IgG2, IgG3, IgG4, IgA1, and IgA2 or subclass of immunoglobulin molecules. Further, the terms “antibody” and “immunoglobulin” can be used interchangeably throughout the specification. Antibodies or immunoglobulins can be used to encompass not only whole antibody molecules, but also antibody multimer, antibody fragments as well as variants of antibodies, antibody multimers and antibody fragments. The immunoglobulin molecules can be isolated from nature or prepared by recombinant means or chemically synthesized. Antibodies and immunoglobulins of the invention can be used for various purposes. In a preferred embodiment, antibodies and immunoglobulins can be used for the detection of the biomarkers.

As used herein, “biomarkers” refers to molecules (e.g., proteins, polypeptides, polynucleotides, oligonucleotides, mRNA, genomic DNA, or DNA transcripts) found in a cell that is correlated with a normal or abnormal condition. In some embodiments of the invention, the term biomarker refers to proteins, polypeptides, polynucleotides, oligonucleotides, mRNA, genomic DNA, and DNA transcripts that are associated with particular cell types and/or particular cancers. Further, biomarker may refer to RNA expression, metabolites, protein expression, or other upstream or downstream mediators. In another embodiment of the invention, the term biomarker refers to the complementary sequence of mRNA or DNA of a biomarker.

As used herein, “positive biomarker(s)” refers to a biomarker that is found in cells associated with a specific disorder, cancer, tumor, and/or condition, and which therefore, either alone or in combination with other biomarkers, indicates or suggests the presence of that specific disorder, cancer, tumor, and/or condition. Positive biomarkers may also refer to biomarkers that are predictive markers of a specific cancer type or stage and thus can be used to indicate the prognosis of a specific disorder, cancer, and/or tumor. The same biomarker may be a positive biomarker for one specific cancer and a negative biomarker for a different specific cancer.

As used herein, “negative biomarker(s)” refers to a biomarker that is found in certain types of cells, but is not found in cells associated with a specific disorder, cancer, tumor, and/or condition being tested for. The same biomarker may be a positive biomarker for one specific cancer and a negative biomarker for a different specific cancer. Negative biomarkers may be used as an internal control and/or an external control.

The term “measuring the expression,” as used herein, refers to measuring the expression of biomarker(s) using techniques commonly used by those skilled in the art. The expression may be measured, for example, at the nucleic acid or protein level. In specific embodiments of the invention, measuring the expression of biomarker(s) is through Immunohistochemistry. In other embodiments of the invention, measuring the expression of biomarker(s) is through in situ hybridization.

The term “plurality,” as used herein, refers to more than one. In specific embodiments of the invention, plurality of biomarkers refers to more than one biomarkers.

The term “proteins” and “polypeptides” are used interchangeably herein and are intended to include any fragments thereof, including, in some particular embodiments, immunologically detectable fragments.

The term “diagnosis” refers to methods by which one skilled in the art can estimate and/or determine whether or not a patient is suffering from, or is at some level of risk of developing, a given disease or condition.

Expression of a particular marker is discussed herein in terms of both the distribution of cells positive for the marker and the intensity with which cells stain for a particular marker.

Distribution of expression relates to the percentage of cells which are positive for a particular marker. A distribution of “4+” indicates that more than about 75% of cells in a particular population show measured expression of the specific biomarker. A distribution of “3+” indicates that about 51% to about 75% of cells in a particular population show measured expression of the specific biomarker. A distribution of “2+” indicates that about 25% to about 50% of cells in a particular population show measured expression of the specific biomarker. A distribution of “1+” indicates that less than about 25% of cells in a particular population show measured expression of the specific biomarker.

With regard to staining intensity, a marker that stains with Strong intensity (“S”) can readily be visualized utilizing a microscope objective of 5× or less, for example a 2× or 4× objective lens. A marker that stains with Weak intensity (“W”) requires a microscopic objective greater than 15× to be readily visualized on cells, for example a microscopic objective of 20× or 40×. A marker that stains with Intermediate intensity (“I”) can be readily visualized with a microscopic objective between 5× and 15×, for example a 10× objective lens.

The term “high-positive cell line(s),” as used herein, refers to a cell line that demonstrates a high level of expression of a specific positive biomarker(s) in that about 51% or more of the cells for a particular cell line show measured expression of the specific positive biomarker (i.e., distribution of 3+ or 4+) and the intensity of expression of the specific positive biomarker(s) in the cells is Intermediate (I) or Strong (S). In other words, high-positive cell line(s) would have a distribution of 3+ or 4+ and an intensity of I or S of the specific positive biomarker(s).

The term “low-positive cell line(s),” as used herein, refers to a cell line that demonstrates a low level of expression of specific positive biomarker(s) in that some portion of the cells for the particular cell line(s) show measured expression of the specific positive biomarker(s) (i.e., distribution of 1+, 2+, 3+, or 4+) and the intensity of expression of the specific positive biomarker(s) in the cells is Weak (W). In other words, low-positive cell line(s) would have a distribution of 1+, 2+, 3+, or 4+ and an intensity of W of the specific positivity biomarker(s).

The term “null-positive cell line(s),” as used herein, refers to a cell line that demonstrates null expression of specific positive biomarker(s). “Null expression” indicates substantially no detectable expression of the given biomarker, or expression that is below the threshold required to qualify as low level expression, as defined above.

The term “high-negative cell line(s),” as used herein, refers to a high level of expression of a specific negative biomarker(s) in that about 51% or more of the cells for a particular cell line(s) show measured expression of the specific negative biomarker(s) (i.e., distribution of 3+ or 4+) and the intensity of expression of the specific negative biomarker(s) is Intermediate (I) or Strong (S). In other words, high-negative cell line(s) would have a distribution of 3+ or 4+ and an intensity of I or S for the specific negative biomarker(s).

The phrase “Tissue Microarray (TMA) block,” as used herein, refers to embedded tissue and/or cells that may be used for IHC analysis. In specific embodiments of the invention, TMA blocks comprise multiple cell lines embedded in paraffin. In other embodiments of the invention, the TMA blocks may comprise any embedding material used by those skilled in the art.

### Method for Producing IHC Control TMA Blocks

The present invention pertains to methods for producing TMA blocks from a mixture of cultured cell lines for use as an IHC control block. In one aspect, the method comprises (A) identifying a plurality of positive biomarkers useful in diagnosis and/or prognosis of one or more particular cancers, and, optionally, measuring the expression of said positive biomarkers in two or more cell lines; (B) selecting: (i) one or more high-positive cell lines that each have a high level of expression of one or more of the plurality of positive biomarkers such that the high-positive cell lines collectively provide high level expression of all of said plurality of positive biomarkers, and one or both of: (ii) one or more low-positive cell lines that each have a low level of expression of one or more of the plurality of positive biomarkers, and (iii) one or more null-positive cell lines that each have null expression of one or more of the plurality of positive biomarkers, wherein a single selected cell line may be from both groups i) and ii); i) and iii); ii) and iii); or i), ii), and iii) for different positive biomarkers; (C) mixing cells of said selected cell lines; and (D) producing a TMA block from said mixed cells.

In some embodiments of the invention, the TMA block comprises two or more cell lines. In other embodiments of the invention, the TMA block comprises three, four, five, six, seven, eight, nine, 10, 11, 12, 13, 14, 15, 16, 17, 18, 19, or more cell lines. In further embodiments of the invention, the TMA block comprises twenty or more cell lines.

In certain embodiments of the invention, the produced TMA block is useful for diagnosing one or more particular cancers. In other embodiments of the invention, the produced TMA block is useful for diagnosing two, three, four, five, six, seven, eight, nine, or more particular cancers. In further embodiments of the invention, the produced TMA block is useful for diagnosing ten or more particular cancers. In yet other embodiments of the invention, the produced TMA block is useful for diagnosing one or more of carcinoma, melanoma, breast cancer, lymphoma, hematopoietic tumor, germ cell tumor, malignant small round cell, blue cell tumor, sarcoma, spindle cell neoplasm, and a tumor of unknown primary origin.

In certain embodiments of the invention, the plurality of positive biomarkers comprises two or more biomarkers. In other embodiments of the invention, the plurality of positive biomarkers comprises three, four, five, six, seven, eight, nine, or more biomarkers. In further embodiments of the invention, the plurality of positive biomarkers comprises 10 or more biomarkers.

Measuring the expression of biomarker(s) may be accomplished through a variety of techniques known to those skilled in the art. In certain embodiments of the invention, measuring the expression of biomarker(s) is done through immunohistochemical techniques. In other embodiments of the invention, measuring the expression of biomarker(s) is done through in situ hybridization. In further embodiments of the invention, measuring the expression of biomarker(s) is accomplished with antibodies that bind to biomarker(s).

In certain embodiments of the invention, the low-positive cell lines collectively provide low level expression of the majority of said plurality of positive biomarkers. In other embodiments of the invention, the low-positive cell lines collectively provide low level expression of all of said plurality of positive biomarkers. In further embodiments of the invention, the null-positive cell lines collectively provide null expression of the majority of said positive biomarkers. In yet other embodiments of the invention, the null-positive cell lines collectively provide null expression of all of said positive biomarkers.

In some embodiments of the invention, the method further comprises identifying one or more negative biomarkers useful in diagnosis and/or prognosis of one or more particular cancers; optionally measuring the expression of said negative biomarkers; and selecting one or more high-negative cell lines that each have a low level or null expression of positive biomarkers and a high level of expression of one or more of the negative biomarkers; wherein a single selected cell line may be both a high-negative cell line and a null-positive cell line, or a single selected cell line may be both a high-negative cell line and a low-positive cell line.

In certain embodiments of the invention, one or more negative biomarkers are identified. In other embodiments of the invention, two, three, four, five, six, seven, eight, nine, or more negative biomarkers are identified. In further embodiments of the invention, 10 or more negative biomarkers are identified.

In some embodiments of the invention, the high-negative cell lines collectively provide high level expression of the majority of said negative biomarkers. In other embodiments of the invention, the high-negative cell lines collectively provide high level expression of all of said negative biomarkers.

In certain embodiments of the invention, the method further comprises determining the combination of selected cell lines required to create a TMA block. In some embodiments of the invention, the ratio produces a TMA block with between about 20% to about 80% of cells having a high level of expression of each of the plurality of positive biomarkers. In other embodiments of the invention, the ratio produces a TMA block with between about 20% to about 80% of cells having a low level of expression of one or more of the plurality of positive biomarkers. In further embodiments of the invention, the ratio produces a TMA block with between about 20% to about 80% of cells having null expression of one or more of the plurality of positive biomarkers. In yet other embodiments of the invention, the ratio produces a TMA block with between about 20% to about 80% of cells having a high level of expression of each of one or more of the plurality of negative biomarkers.

In certain embodiments of the invention, the ratio produces a TMA block with greater than about 20% of cells having a high level of expression of each of the plurality of positive biomarkers. In other embodiments of the invention, the ratio produces a TMA block with between about 30% to about 70% of cells having a high level of expression of each of the plurality of positive biomarkers. In further embodiments of the invention, the ratio produces a TMA block with between about 40% to about 60% of cells having a high level of expression of each of the plurality of positive biomarkers. In yet other embodiments of the invention, the ratio produces a TMA block with less than about 80% of cells having a high level of expression of each of the plurality of positive biomarkers.

In certain embodiments of the invention, the ratio produces a TMA block with greater than about 20% of cells having a low level of expression of one or more of the plurality of positive biomarkers. In other embodiments of the invention, the ratio produces a TMA block with between about 30% to about 70% of cells having a low level of expression of one or more of the plurality of positive biomarkers. In further embodiments of the invention, the ratio produces a TMA block with between about 40% to about 60% of cells having a low level of expression of one or more of plurality of the positive biomarkers. In yet other embodiments of the invention, the ratio produces a TMA block with less than about 80% of cells having a low level of expression of one or more of the plurality of positive biomarkers.

In certain embodiments of the invention, the ratio produces a TMA block with greater than about 20% of cells having no expression of each of the plurality of positive biomarkers. In other embodiments of the invention, the ratio produces a TMA block with between about 30% to about 70% of cells having no expression of each of the plurality of positive biomarkers. In further embodiments of the invention, the ratio produces a TMA block with between about 40% to about 60% of cells having no expression of each of the plurality of positive biomarkers. In yet other embodiments of the invention, the ratio produces a TMA block with less than about 80% of cells having no expression of each of the plurality of positive biomarkers.

In certain embodiments of the invention, the ratio produces a TMA block with greater than about 20% of cells having a high level of expression of each of the plurality of negative biomarkers. In other embodiments of the invention, the ratio produces a TMA block with between about 30% to about 70% of cells having a high level of expression of each of the plurality of negative biomarkers. In further embodiments of the invention, the ratio produces a TMA block with between about 40% to about 60% of cells having a high level of expression of each of the plurality of negative biomarkers. In yet other embodiments of the invention, the ratio produces a TMA block with less than about 80% of cells having a high level of expression of each of the plurality of negative biomarkers.

### TMA Blocks for Use as a IHC Control Block

The present invention also pertains to TMA blocks for use in diagnosis and/or prognosis of one or more particular cancers. In certain embodiments of the invention, the TMA block is a Melanoma Control Block comprising G361 cells, CRL-1585 cells, and CRL-5895 cells, wherein total cells used are about 40% of said G361 cells, about 40% of said CRL-1585 cells, and about 20% of said CRL-5895 cells. In other embodiments of the invention, the TMA block is a Breast Cancer Control Block comprising HTB133, CRL-2330, and CCL-253 cells, wherein total cells used are about 40% of said HTB-133 cells, about 40% of said CCL-253 cells, and about 20% of said CRL-2330 cells. In further embodiments, the TMA block is a Lymphoma/Hematopoietic Tumor Control Block comprising CRL-1582-Molt4, CCL-86-BLM, and CRL-5895 cells, wherein total cells used are about 40% of said CRL-1582-Molt4 cells, about 40% of said CCL-86-BLM cells, and about 20% of said CRL5895 cells. In yet other embodiments of the invention, the TMA block is a Germ Cell Tumor Control Block comprising CRL-2073, HTB-36, HepG2, and CRL-1585 cells, wherein total cells used are about 25% of said CRL-2073 cells, about 25% of said HTB-36 cells, about 25% of said HepG2 cells, and about 25% of said CRL-1585 cells. In other embodiments of the invention, the TMA block a Malignant Small Round Cell/Blue Cell Tumor Control Block comprising CCL-136, HTB166, TT, CRL-5946, CRL-1582-Molt4, and CCL-86-BLM cells, wherein total cells used are about 20% of said CCL-136 cells, about 20% of said HTB166 cells, about 20% of said TT cells, about 20% of said CRL-5946, about 10% of said CRL-1582-Molt4, and about 10% of said CCL-86-BLM cells. In certain embodiments of the invention, the TMA block is a Sarcoma/Spindle Cell Neoplasm Control Block comprising HTB166, CCL-136, CRL-2279, CRL-1585, and CRL-1550 cells, wherein total cells used are about 20% of said HTB166 cells, about 20% of said CRL-136 cells, about 20% of said CRL-2279 cells, about 20% of said CRL-1585 cells, and about 20% of said CRL-1550 cells. In further embodiments of the invention, the TMA block is a Tumor of Unknown Primary Control Block comprising HTB133, NCI-H508, TT, Pan 3.27, CRL-1932, CRL-2279, CRL-1550, and CRL-5946 cells, wherein total cells used are about 15% of said HTB133 cells, about 15% of said NCI-H508 cells, about 20% of said TT cells, about 10% of said Pan3.27 cells, about 10% of said CRL-1932 cells, about 10% of said CRL-2279 cells, about 10% of said CRL-1550 cells, and about 10% of said CRL-5946 cells.

In certain embodiments of the invention, the TMA block is a Melanoma Control Block comprising cells of a plurality of cultured cell lines, wherein said cultured cell lines collectively provide high-level expression and no expression of positive biomarkers S100, HMB45, MiTF, Mart-1, SOX10, SOX2, MUM1, S100A6, and Vimentin; low-level expression of a plurality of biomarkers S100, HMB45, MiTF, Mart-1, SOX10, SOX2, MUM1, S100A6, and Vimentin; and high-level expression and no expression of negative biomarkers Cytokeratin and Cytokeratin 7. In other embodiments of the invention, the TMA block is a Breast Cancer Control Block comprising cells of a plurality of cultured cell lines, wherein said cultured cell lines collectively provide high level expression, low level expression and no expression of biomarkers HER2, estrogen receptors (ER), progesterone receptors (PR), GATA3, GCDPF15, mammaglobin, TFF1, TFF3, and CK7. In further embodiments of the invention, the TMA block is a Lymphoma/Hematopoietic Tumor Control Block comprising cells of a plurality of cultured cell lines, wherein said cultured cell lines collectively provide high level expression, low level expression and no expression of biomarkers CD2, CD3, CD5, CD7, CD10, CD20, CD79a, PAX5, Bcl2, Bcl6, EBV, TdT, CD99, CK and CK7. In yet other embodiments of the invention, the TMA block is a Germ Cell Tumor Control Block comprising cells of a plurality of cultured cell lines, wherein said cultured cell lines collectively provide high level expression, low level expression and no expression of biomarkers SALL4, OCT4, PLAP, beta-HCG, alpha-fetoprotein, glypican 3, D2-40, CD30, SOX2, Nanog, S100P, and cytokeratin. In certain embodiments of the invention, the TMA block is a Malignant Small Round Cell/Blue Cell Tumor Control Block comprising cells of a plurality of cultured cell lines, wherein said cultured cell lines collectively provide high level expression, low level expression and no expression of biomarkers desmin, MyoD1, myogenin, smooth muscle actin, CD99, NKX2.2, Fli-1, synaptophysin, chromogranin, CD56, NSE, WT-1, vimentin, TTF1, cytokeratin, CD2, CD3, TdT, CD20, CD79a, and EBV. In further embodiments of the invention, the TMA block is a Sarcoma/Spindle Cell Neoplasm Control Block comprising cells of a plurality of cultured cell lines, wherein said cultured cell lines collectively provide high level expression, low level expression and no expression of biomarkers desmin, MyoD1, myogenin, smooth muscle actin, CD99, NKX2.2, Fli-1, ERG, vimentin, S100, CK5/6, CK903, p63, p40, p16, and cytokeratin. In yet other embodiments of the invention, the TMA block is a Tumor of Unknown Primary Control Block comprising cells of a plurality of cultured cell lines, wherein said cultured cell lines collectively provide high level expression, low level expression and no expression of biomarkers pan cytokeratin, CK7, CK20, CK5/6, CK903, p63, p40, ER, PR, GATA3, GCDFP15, CDX2, SATB2, cadherin-17, CEA, MOC31, BerEP4, beta-cateinin, B72.3, arginase-1, HepPar1, TTF1, calcitonin, chromogranin, synaptophysin, CD56, MUC1, MUC2, MUC5AC, maspin, S100P, PAX2, PAX8, vimentin, P504S, ERG, Fli-1, p16, WT-1, and HPV (in situ).

The present invention also pertains to a Universal Tissue Microarray Block for use as an IHC control block in differential diagnosing. In certain embodiments of the invention, the Universal Tissue Microarray Block is used in differential diagnosing of one or more of carcinoma, melanoma, germ cell tumor, sarcoma, and lymphoma. In further embodiments of the invention, the Universal Tissue Microarray Block comprises G361, CRL-1585, CRL-1582-Molt4, CCL-86-BLM, CRL-2073, HTB-36, HepG2, CCL-136, HTB166, HTB133, CCL-253, NCI-H508, TT, Pan 3.27, CRL-1932, CRL-2279, CRL-1550, and CRL-5946 cells. In further embodiments, the Universal Tissue Microarray Block comprises cell lines wherein each cell line comprises from about 1% to about 25% of the total cells of the Universal Tissue Microarray Block. In some embodiments of the invention, each cell line comprises from about 1% to about 20% of the total cells of the Universal Tissue Microarray Block. In other embodiments of the invention, each cell line comprises from about 1% to about 15% of the total cells of the Universal Tissue Microarray Block. In further embodiments of the invention, each cell line comprises from about 2% to about 10% of the total cells of the Universal Tissue Microarray Block. In yet other embodiments of the invention, each cell line comprises from about 1% to about 5% of the total cells of the Universal Tissue Microarray Block.

In other embodiments of the invention, the cell lines of the Universal Tissue Microarray Block collectively provide high level expression, low level expression, and no expression of biomarkers. In further embodiments of the invention, the biomarkers may include S100, HMB45, MiTF, Mart-1, SOX2, SOX10, SALL4, OCT4, PLAP, beta-HCG, AFP, glypican 3, CD30, D2-40, HER2, ER, PR, GATA3, GCDFP15, MGB, CK7, CK20, CK5/6, CK903, p40, p63, CDX2, SATB2, cadherin-17, TTF1, napsin A, calcitonin, chromogranin, synaptophysin, CD56, MUC1, MUC2, MUC5AC, calretinin, MOC31, BerEP4, Arginase-1, HepPar-1, PAX2, PAX8, IMP3, maspin, vimentin, p16, WT-1, HPV, desmin, MyoD1, myogenin, SMA, NKX2.2, Fli-1, ERG, CD2, CD3, CD5, CD10, CD20, CD79a, PAX5, TdT, EBV, Ki-67, and/or p53.

The present invention pertains to the construction of TMA blocks from the cultured cell lines. In some embodiments of the invention, the construction of TMA blocks is done in the following steps: 1) culture the cell lines, for example cell lines obtained from the American Type Culture Collection (ATCC); 2) build a cell block from each cultured cell line; 3) test the selected biomarkers/antibodies on the constructed cell block; 4) mix the selected cell lines in desired particular ratio to provide desired expression levels of positive and negative biomarkers; 5) construct tissue microarray (TMA) blocks from the cell blocks containing selected mixed cell lines; and 6) re-test the selected antibodies on the constructed TMA blocks to confirm the expression of the targeted biomarkers.

### Examples

### Example 1: Culture of Cell Lines

Cells and cell cultures are maintained by common techniques know to those skilled in the art. Further, cell lines may be obtained from any suitable source, such as the American Type Culture Collection (ATCC, Manassas, Va.), from which the cell lines utilized in these examples were purchased. The ordering information, growth conditions, and properties of the cell lines utilized herein are summarized in Table 1.

Upon arrival of the frozen cell lines from the ATCC, the vial containing each cell line was thawed by gentle agitation in a 37° C. water bath. Thawing took approximately 2 minutes. The vial was removed from the water bath as soon as the contents were thawed and the vial was decontaminated by spraying with 70% ethanol.

Each vial containing a specific cell line was handled in an appropriate manner based upon the cell growth property, as set forth below:

Cell lines with suspended growth:
a. Transfer the vial contents to a centrifuge tube containing 9.0 ml complete culture medium and spin at approximately 125×g for 5 to 10 minutes to pellet cells;
b. Re-suspend the cell pellet with the recommended complete medium and transfer into a 100×20 mm tissue culture dish (Cat. #83.1802, Sarstedt, Newton N.C.);
c. Incubate the cell culture at 37° C. in a CO₂ incubator (Thermo Forma) until the cells are ready to be sub-cultured.

Cell lines with adherent growth:
a. Transfer the vial contents directly to a 100×20 mm tissue culture dish with the recommended complete medium;
b. When the cells show adherent growth in the dish, replace the medium with fresh complete medium and incubate the culture dish at 37° C. in a CO₂ incubator until cells are ready to be sub-cultured.

A 5% CO₂ in air atmosphere was used unless the culture must be CO₂ free when a special culture medium such as Leibovitz's L-15 was applied. Table 1 summarizes the growth features of the cell lines utilized in this example.

Following initial culturing, the cells were subcultured as follows:

Subculture of cell lines with adherent growth:
a. Remove and discard the culture medium;
b. Briefly rinse the cell layer with 10 mM PBS;
c. Add 1.0 ml of 0.25% (w/v) trypsin in 0.53 mM EDTA solution to the dish and observe the cells under an inverted microscope until the cell layer is dispersed (usually within 1 to 3 minutes);
d. Add 6.0 ml to 8.0 ml of complete growth medium and aspirate the cells by gently pipetting;
e. Add appropriate aliquots of the cell suspension to two 150×25 mm tissue culture dishes (Cat. #25383-103, VWR, Bridgeport N.J.) for the new sub-culturing.

Subculture of cell lines with suspended growth:
a. Transfer the dish contents to a 50 ml centrifuge tube and spin down;
b. Re-suspend the cell pellet with fresh complete medium and transfer into two 150×25 mm tissue culture dishes for the new sub-culturing.

Cultures were established between 2×10³ and 1×10⁴ viable cells/cm². Cultures were maintained at a cell concentration less than 1˜5×10⁷ per 150×25 mm tissue culture dish. A sub cultivation ratio was 1:3 to 1:10 depending on the cell doubling time. A cell counter (Cellometer Auto T4, Nexcelon Bioscience) was used to count the numbers of cells.

TABLE 1
Ordering information, growth condition, and growth properties of cultured cell lines\hat{ }

| ATCC Cat. No. | Designation | Source | Growth Condition and Growth Properties |
|---|---|---|---|
| CCL-253 | NCI-H508 | Colorectal adenocarcinoma | RPMI160 + 10% FBS |
| CCL-218 | WiDr | Colon adenocarcinoma | EMEM + 10% FBS |
| CCL-229 | LoVo | Colon Adenocarcinoma | F12K + 10% FBS |
| HTB-37 | Caco-2 | Colon adenocarcinoma | EMEM + 10% FBS |
| CCL-231 | SW48 | Colon adenocarcinoma | Leibovitz's L-15 + 10% FBS, CO2 free |
| CCL-221 | DLD-1 | Colon adenocarcinoma | RPMI 1640 + 10% FBS |
| CRL-5822 | NCI-N87 | Gastric carcinoma | RPMI 1640 + 10% FBS |
| CRL-2547 | Panc 10.05 | Pancreatic adenocarcinoma | RPMI + 15% FBS + human insulin10 unite/ml |
| CRL-2549 | Pane 03.27 | Pancreatic adenocarcinoma | RPMI + 15% FBS + human insulin10 unite/ml |
| CRL-2551 | Panc 08.13 | Pancreatic adenocarcinoma | RPMI + 15% FBS + human insulin10 unite/ml |
| CRL-2279 | MS1 | Pancreas/islet of Langerhans; | DMEM + 10% FBS |
| | | endothelium(mouse) | |
| CRL-2237 | SNU-387 | Pleomorphic hepatocellular carcinoma | RPMI 1640 + 10% FBS |
| CRL-2234 | SNU-449 | Hepatocellular carcinoma (HBV+) | RPMI 1640 + 10% FBS |
| CRL-11233 | THLE-3 | Liver epithelial SV40 transformed, normal | BEGM + additives* + 10% FBS |
| CRL-2706 | THLE-2 | Liver epithelial SV40 transformed, normal | BEGM + additives* + 10% FBS |
| CRL-1830 | Hepa1-6 | Hepatocellular carcinoma (mouse) | DMEM + 10% FBS |
| HB-8065 | HepG2 | Hepatocellular carcinoma | EMEM + 10% FBS |
| CRL-10741 | C3A | Hepatocellular carcinoma | EMEM + 10% FBS |
| CRL-11268 | 293T/17 | Fetus kidney SV-40 | DMEM + 10% FBS |
| CRL-1611 | ACHN | Renal cell adenocarcinoma | EMEM + 10% FBS |
| CRL 1932 | 786-O | Renal cell adenocarcinoma | RPMI 1640 + 10% FBS |
| HTB-47 | Caki-2 | Kidney clear cell Carcinoma | McCoy's 5a + 10% FBS |
| CRL-1441 | G401 | Rhabdoid tumor - Wilms tumor | McCoy's 5a + 10% FBS |
| CRL-1435 | PC-3 | Prostate adenocarcinoma | F-12K + 10% FBS |
| CRL-2505 | 22Rv1 | Prostate carcinoma PSA+ | RPMI 1640 + 10% FBS |
| PCS-440-010 | N/A | Prostate(normal) | ATCC-PCS-440-030; 440-040; 30-2200 |
| CCL-185 | A549 | Lung carcinoma | F-12K + 10% FBS |
| HTB-184 | NCL-H510A | Lung small cell cancer | F-12K + 10% FBS, adherent and suspension |
| CCL-256 | NCI-H2126 | Lung adenocarcinoma | HITES** + 5% FBS |
| CRL-5826 | NCI-H226 | Lung squamous cell carcinoma; mesothelioma | RPMI 1640 + 10% FBS |
| CRL-5877 | NCI-H1573 | Lung adenocarcinoma | RPMI1640 + 5% FBS |
| CRL-5895 | NCI-H1792 | Lung adenocarcinoma | RPMI1640 + 10% FBS |
| CRL-5908 | NCI-H1975 | Lung non-small cell carcinoma | RPMI1640 + 10% FBS |
| CRL-5869 | NCI-H1417 | Classic small cell lung cancer | RPMI_1640 + 10% FBS, suspension |
| CCL-257 | NCL-H1688 | Lung carcinoma, classic small cell lung cancer | RPMI 1640 + 10% FBS |
| HTB-177 | H-460 | Carcinoma; large cell lung cancer | RPMI 1640 + 0% FBS |
| CRL-1596 | Ramos(RA 1) | Burkitt's lymphoma | RPMI 1640 + 10% FBS suspension |
| CRL-2974 | MM.1S | B Lymphoblast myeloma | RPMI 1640 + 10% FBS suspension and lightly |
| | | | attached |
| CCL-159 | IM-9 | B lymphoblast, Epstein Barr virus(EBV) | RPMI 1640 + 10% FBS suspension |
| | | transformed | |
| CCL-86 | Raji | Burkitt's Lymphoma | RPMI 1640 + 10% FBS suspension |
| CRL-1582 | MOLT4 | Acute lymphoblastic leukemia | RPMI 1640 + 10% FBS suspension |
| CC1-119 | CCRF-CEM | Leukemia T-cell human | RPMI 1640 + 10% FBS, suspension |
| CRL-2264 | CEM/C2 | Leukemia, T-cell human | RPMI 1640 + 10% FBS, suspension |
| CRL-2294 | BCP-1 | B Lymphoblast lymphoma | RPMI 1640 + 20% FBS, suspension |
| CRL-1585 | C32 | Melanoma | EMEM + 10% FBS |
| CRL-1424 | G361 | Melanoma | McCoy's + 10% FBS |
| CRL-1872 | A375.S2 | Melanoma | EMEM + 10% FBS |
| CRL-2329 | HCC1500 | Primary ductal carcinoma (ER+/PR+/HER−) | RPMI1640 + 10% FBS |
| HTB-133 | T-47D | Breast ductal carcinoma (ER+/PR+) | RPMI1640 + 10% FBS |
| CRL-2330 | HCC1569 | Breast primary metaplastic carcinoma (HER2+) | RPMI1640 + 10% FBS, suspension and adherent |
| CRL-2321 | HCC1143 | Breast carcinoma(ER−/PR−/HER−) | RPMI1640 + 10% FBS |
| CRL-1902 | UACC893 | Breast primary ductal carcinoma | Leibovitz's L-15 + 10% FBS, CO2 free |
| HTB-36 | JEG-3 | Placenta choriocarcinoma | EMEM + 10% FBS |
| HTB-105 | Tera-1 | Embryonal carcinoma malignant | McCoy's 5a + 10% FBS |
| CRL-2073 | NCCIT | Pluripotent embryonal carcinoma; | RPMI 1640 + 10% FBS |
| | | Teratocarcinoma | |
| CRL-2180 | L2-RYC | Yolk sac carcinoma | DMEM + 10% FBS |
| HTB166 | RD-ES | Ewing's sarcoma | RPMI 1640 + 15% FBS |
| CRL-7556 | Hs-822.T | Ewing's sarcoma | DMEM + 10% FBS |
| CCL-136 | RD | Rhabdomyosarcoma | DMEM + 10% FBS |
| CRL-7822 | Hs 5.T | Leiomyosarcoma | RPMI 1640 + 10% FBS |
| HTB-93 | SW-982 | Synovial sarcoma | Leibovitz's L-15 + 10% FBS, CO2 free |
| CRL-11882 | Hs-5 | Stromal Cells | DMEM + 10% FBS |
| CRL-2946 | UWB1.289 + BRCA1 | Ovarian carcinoma | 50% RPMI-1640 + 50% MEGM*** + 3% FBS |
| CRL-1550 | Ca-ski | Cervical carcinoma | RPMI 1640 + 10% FBS |
| HTB35 | SiHa | Cervical carcinoma | EMEM + 10% FBS |
| CRL-5946 | NCI-H2452 | Mesothelioma | RPMI 1640 + 10% FBS |
| CRL-5946 | NCI-H2452 | Mesothelioma | RPMI 1640 + 10% FBS |
| CRL-1803 | TT | Thyroid/Medullary Carcinoma | F12 + 20% FBS |
| CRL-10296 | NCL-H295 | Adrenocortical carcinoma steroid hormones+ | HITES** + 2% FBS |

*BEGM (Lonza) CC-3170 Kit: BEGM Bullet Kit (CC-3171 & CC-4175) CC-3171: Basal Medium - contains no growth factors, cytokines, or supplements CC-4175: SingleQuots ™ Kit - growth factors, cytokines, and supplements
**HITES HITES medium supplemented with 5% FBS The base medium DMEM:F12 Medium, ATCC 30-2006 To make the complete growth medium, add the following components to the base medium 1. 0.005 mg/ml insulin 2. 0.01 mg/ml transferrin 3. 30 nM sodium selenite (final conc.) 4. 10 nM hydrocortisone (final conc.) 5. 10 nM betaestradiol (final conc.) 6. extra 2 mM L-glutamine (for final conc. of 4.5 mM) 7. 5% fetal bovine serum (final conc.)
***(MEGM (Lonza) CC-3150: MEGM BulletKit (CC-3151 & CC-4136) CC-3151: MEBM Basal Medium 500 ml CC-4136: MEGM SingleQuot Kit Suppl. & Growth Factors
****MCBD 105: Medium 199 Sigma Aldrich M6395: Life Technologies 11150-059
Other Medium:
RPMI1640: Life Technologies 11875119;
DMEM: Life Technologies 11995073;
McCoy's 5A: ATCC 30-2007;
Leibovitz's L-15: ATCC 30-2008;
DMEM-F12 medium: ATCC 30-2006;
Eagle's minimum essential medium: ATCC 30-2003
\hat{ }Growth properties: all cell lines were adherent growth unless otherwise mentioned in the table.

### Example 2: Cryopreservation of the Cultured Cells for Future Use

Cultured cells were cryopreserved using standard techniques known to those in the art. The following is a brief example:

1.5 ml low temperature freezer vials were used to cryopreserve the cells. 2×10⁵ to 1×10⁶ cells together with 50% FBS, 40% medium and 10% (v/v) DMSO were included in each vial. Place the vials in the Cryo-Safe™ −1° C. freeze controller (Cat. #18844-000, Bel-Art, Wayne N.J.), which was filled with 250 ml of 100% isopropyl alcohol. Once the vials containing cells had been inserted into the controller, we placed the controller in a −80° C. mechanical freezer. The cells could be stored in a −80° C. mechanical freezer for several weeks if needed. The vials were then transferred into a Cryostar liquid nitrogen freezer (−140° C.) for permanent preservation or future use.

### Example 3: Cell Harvest and Semi-Solid Cell Pellet Preparation

Cells were harvested and cell pellets were prepared using standard techniques known to those in the art. The following is a brief example:

When the cell growth was near confluent (adherent growth, about 1×10⁷ cells per dish) or near 0.5×10⁸ cells per dish (suspended growth), harvest cells by EDTA digestion and centrifuge (adherent growth); or directly move the suspended growth cultures to 50 ml Falcon tubes. Eight large culture dishes (150×25 mm each; each dish containing 5-10×10⁷ cells) were collected for one cell block preparation.

For the preparation of a cell block with mixed cell lines, we cultured select cell lines simultaneously and mixed these cells at proper ratios depending on the purpose of the cell block. Table 2 shows an example of the melanoma control block with three different cell lines in the proper ratios and cell counts.

An example for the steps to prepare for a cell pellet are as follows:
a. Centrifuge the cells down to make a cell pellet.
b. Move the bottom cell pellet to a small glass tube (Cat. #72631-10, Electron Microscopy Sciences, Hatfield, Pa.), then place the small glass tube to a 25 ml Polyethylene Vial (Cat. #72621-62, Electron Microscopy Sciences, Hatfield, Pa.).
c. Spin down the cells at 1600 rpm for 7 minutes in a Beckman centrifuge with Swinging-Bucket Rotors.
d. Remove the small glass tube from centrifuge for cell block preparation.
e. Pour off supernatant completely, but preserve the cell pellet at bottom of the small tube.
f. Add approximately 7 drops of plasma (From Blood Bank of Geisinger Clinic) to the cell pellet and re-suspend by gently vortexing; then add approximately 7 drops of bovine thrombin (Cat. #23-306291, Fisher Scientific) into the cell pellet and mix gently, then let it stand for 10 minutes.
g. The cell pellet should become a semi-solid clot at room temperature. Under a fume hood, insert a 23 gauge needle with the syringe which includes approximately 6 ml of 10% Neutral-buffered formalin along the side at the bottom of the tubes. While the formalin was slowly pushed through the syringe, the rounded cell pellet is slowly dislodged from the flat bottom glass tube and floated to the surface of the small tube.
h. Place the clotted pellet into a labeled cassette. Transfer the cassette into a 10% Neutral-Buffered formalin container, and then transport it to the histology lab for the tissue processing and paraffin embedding. The cell clotted pellet will be fixed in 10% formalin for at least 8 hours but less than 24 hours.

TABLE 2
Cell lines for construction of melanoma control block

| | Ratio of each cell | |
|---|---|---|
| ATCC Cat. # | Line | Total cells per block |
| CRL-1585 | about 40% | about 2.50 × 10⁷ |
| CRL-1424 | about 40% | about 2.50 × 10⁷ |
| CRL-5895 | about 20% | about 1.25 × 10⁷ |

### Example 4: Preparation of Cell Blocks

Cell blocks were prepared using standard techniques known in the art. The following is an example:

After 8-24 hours fixation in 10% formalin, we placed the cassette with the clotted cell pellet in the Tissue Processor to be processed as other routine surgical pathology specimens. After the processing, the clotted cell pellet was embedded with 57-59° C. paraffin on the Embedding Workstation (Cat. # A81000002, HistoStar™, Thermo Scientific). At this step; the diameter of the clotted cell pellet was about 1.0 cm.

Cut the cell block into 4˜5 μm sections, check the quality of the cell block with Hematoxylin and eosin stain (H&E) stain.

### Example 5: Immunohistochemical Detection of Select Biomarkers on Cells in Blocks Containing Single Cell Line, in Blocks Containing Mixed Cell Lines, or in Cultured Cells

Immunohistochemical detection of the select biomarkers was performed on a group of constructed cell blocks containing a single cell line using standard techniques known to those skilled in the art. Table 3 summarizes the measured expression of selected biomarkers on the group of constructed cell blocks containing a single cell line. Selected biomarkers include positive biomarkers for particular cell types and/or specific cancers. Selected biomarkers also include certain negative biomarkers.

Immunohistochemical detection of the select biomarkers was also performed on a group of cell blocks containing the mixed cell lines using standard techniques known to those skilled in the art. Table 4 summarizes the measured expression of selected biomarkers on a group of cell blocks containing mixed cell lines. Selected biomarkers include positive biomarkers for particular cell types and/or specific cancers. Selected biomarkers also include certain negative biomarkers.

Numerous commonly used diagnostic biomarkers were also tested in the cultured cell lines using standard techniques known to those skilled in the art. The antibody ordering information and staining protocols for an example set of antibodies are summarized in Table 5.

Immunohistochemical stains may also be done on cell lines directly, which may serve as a positive control especially for a fine needle aspiration/cytologic specimen.

ATCC does not provide information on the expression of specific biomarkers for each cell line. As demonstrated by Tables 3 and 4, a large number of cell lines were tested to identify suitable cell lines for certain targeted biomarkers. As this shows, the cell lines frequently do not express certain biomarkers which one skilled in the art might expect to be expressed in that cell type. For example, one skilled in the art may incorrectly expect hepatocellular carcinoma cell line or virus-transformed normal liver cell line to express common liver cell biomarkers, such as arginase-1 and HepPar-1. However, when 7 liver cancer and normal liver cell lines were tested, the inventors unexpectedly did not identify any of the 7 cell lines expressing these 2 markers. As another example, TTF1 is an important diagnostic marker for identifying lung adenocarcinoma and lung neuroendocrine carcinoma, and one skilled in the art might incorrectly expect that any ATCC lung adenocarcinoma cell line would express the TTF1 biomarker. However, the inventors unexpectedly found that only one cell line (HTB-184) of the 9 tested lung adenocarcinoma and small cell carcinoma cell lines diffusely and strongly expresses TTF1. The other 8 lung cancer cell lines were found to be either negative for TTF1 or only focally positive for TTF1. Another example that is evident from Tables 3 and 4 in that multiple cell lines were tested to identify a targeted biomarker(s) such as CDX2, CK20, and CEA in the colon cancer cell line (NCI-H508); EBV in the lymphoma cell line (CCL-86); and GATA3 in the breast cancer cell line (HTB-133).

TABLE 3
Summary of positive and negative biomarkers tested in cultured cell lines

| ATCC | | | Tested Positive | Tested Negative |
|---|---|---|---|---|
| Catalog No | Designation | Source | Biomarkers | Biomarkers |
| CRL-2329 | HCC1500 | Breast ductal carcinoma | CK7 (4+, S), AE1/3 (4+, S), | PR(−), HER2 (1+ and −), |
| | | | ER (4+, S), HER2 (1+ and −), | GCDFP15 (−), MGB (−) |
| | | | GATA3 (3+, S) | |
| HTB-133 | T-47D | Breast Ductal carcinoma | CK7 (4+, S), AE1/3 (4+, S), | MGB (−) |
| | | | ER (4+, S), PR (3+, I), HER2 | |
| | | | (1+ and 2+), GATA3 (4+, S), | |
| | | | GCDFP15 (3+, S) | |
| CRL-1902 | UACC-893 | Breast ductal carcinoma | HER2 (3+, S), GATA3 (2+, | ER (−), PR (−), MGB (−) |
| | | | I), TFF1 (3+, I), TFF3 (3+, S), | |
| | | | CK7 (4+, S), p53 (1+, w) | |
| CRL-2330 | HCC1569 | Breast ductal carcinoma | HER2 (3+, S) | ER (−), PR (−) |
| CRL-2321 | HCC1143 | Breast carcinoma | GATA3 (1+, W), MGB (1+, | ER (−), PR (−), TFF1 (−), TFF3 |
| | | | W) | (−), GCDFP15 (−), HER2 (−), |
| CRL-1932 | 786-O | Renal cell carcinoma | PAX8 (4+, S), P5045 (4+, S), | RCC (−), EMA (−), CK20 (−), |
| | | | vimentin (4+, S), pVHL (1+, | CA IX (−), |
| | | | I), CD10 (1+, I) | |
| HTB-47 | Caki-2 | Renal cell carcinoma | PAX8 (4+, S), vimentin (4+, | PAX2 (−), S100 (−), S100A1 |
| | | | S), pVHL (1+, I), P504S (4+, | (−), EMA (−), CK7 (−), CA9 (−), |
| | | | I) | CD10(−), |
| CRL-1611 | ACHN | Renal cell carcinoma | PAX8 (4+, S), PAX2 (4+, I), | RCC (−), CA IX (−), CD10 (−), |
| | | | P504S (3+, S) | EMA (−), Napsin A (−), |
| | | | | S100A1 (−), pVHL (−) |
| CRL-1441 | G401 | Kidney-Wilm's | CD57 (1+, S) | WT1 (−), CD56 (−), |
| | | | | myogenin (−) |
| CRL-11268 | 293T/17 | Fetus kidney SV-40 | BK virus (4+, S) | |
| HTB-37 | CACO-2 | Colon cancer | CK20 (1+, S), CDX2 (3+, S), | B72.3 (−), p53 (−), TFF3 (−), |
| | | | B-catenin (M + N), CEA (1+, | maspin (−), MUC2 (−), |
| | | | I), MOC31 (4+, S), BerEP4 | MUC4 (−) |
| | | | (4+, S), MSI (4+, S), P504S | |
| | | | (3+, S), TFF1 (1+, I), villin | |
| | | | (4+, S), KOC (4+, I) | |
| CCL-221 | DLD-1 | Colon cancer | CK20 (1+, S), CDX2 (4+, I), | CEA (−), B723 (−), P504S (−), |
| | | | B-catenin (N + M), MOC31 | TFF1 (−), TFF3 (−), villin (−), |
| | | | (4+, S), BerEP4 (4+, S), p53 | MUC2 (−), MUC4 (−) |
| | | | (3+, I), MSI (4+, S), maspin | |
| | | | (1+) | |
| CCL-253 | NCI-H508 | Colon cancer | CEA (4+, S), CDX2 (4+, I), | CAIX (−) |
| | | | CK20 (3+, S), B-catenin (4+, | |
| | | | S, M + N), TFF3 (3+, S), villin | |
| | | | (4+, S), TFF-1 (1+, S), p504S | |
| | | | (3+, S) | |
| CCL-218 | WiDr | Colon Cancer | CK20 (4+, S), B-catenin (4+, | CEA (−), CDX2 (−), TFF1 (−), |
| | | | S, M), TFF3 (1+, S), villin | p504S (−) |
| | | | (4+, I), CA IX (3+, S) | |
| CCL-229 | LoVo | Colon cancer | CEA (3+, S), p504S (2+, W), | Villin (−), TFF3 (−), TFF1 (−), |
| | | | CK20 (1+, S), CDX2 (3+, I), | CA IX (−) |
| | | | b-catenin (N + M) | |
| CRL-1424 | G361 | Melanoma | HMB45 (4+, S), SOX10 (4+, | AE1/3 (−) |
| | | | S), Mart-1 (4+, S), | |
| | | | Vimentin (4+, S), S100 (2+, | |
| | | | W), SOX2 (1+, W), S100A6 | |
| | | | (4+, S) | |
| CRL-1585 | C32 | Melanoma | S100 (4+, S), SOX10 (3+, S), | HMB45 (−), AE1/3 (−) |
| | | | Mart-1 (3+, S), MUM1 (3+, | |
| | | | S), S100A6 (4+, S), MITF | |
| | | | (4+, S), SOX2 (3+, S), p53 | |
| | | | (3+, W), vimentin (4+, S) | |
| CRL-1872 | A375.S2 | Melanoma | S100 (4+, S), Mart1 (2+, | HMB45 (−), SOX2 (−) |
| | | | W), SOX10 (2+, S), MUM1 | |
| | | | (1+, S), S100A6 (4+, S), | |
| | | | MITF (4+, S) | |
| CRL-1582 | Molt4 | Lymphoma | Tdt (3+, S), CD3 (4+, S), | CD1a (−), CD20 (−), CD79a |
| | | | CD5 (2+, W), CD2 (2+, S), | (−), CD10 (−), Pax5 (−) |
| | | | CD34 (1+, S), | |
| CCL-86 | Raji | Lymphoma | CD20 (4+, S), CD79a (4+, | Tdt (−), CD3 (−) |
| | | | S), Pax5 (4+, S), CD10 (2+, | |
| | | | I), Ki67 (100%), BCL2 (2+, | |
| | | | W), BCL6 (4+, S), EBV (2+, | |
| | | | S) | |
| CCL159 | IM-9 | B-cell lymphoma | CD20 (3+, S), Tdt | CD3 (−), EBV (−), CD43 (4+, |
| | | | (cytoplasmic (+), CD43 (4+, | S), CD10 (−) |
| | | | S), CD79a (4+, I), PAX5 (4+, | |
| | | | S) | |
| CRL-1596 | Ramos (RA 1) | B-cell lymphoma | CD20 (4+, S), Tdt (cyto +), | CD3 (−), EBV (−), CD5 (−) |
| | | | CD43 (4+, S), CD79a (4+, | |
| | | | S), CD10 (4+, S), PAX5 (4+, | |
| | | | S) | |
| CRL-2974 | MM.1S | Myeloma | Nothing stained | |
| CCL-119 | CCRF-CEM | T-cell lymphoma | CD2 (3+, S, dot stain), CD3 | CD20 (−), CD79a(−), PAX5 (−) |
| | | | (4+, S), CD5 (3+, S), CD10 | |
| | | | (3+, S), CD43 (4+, S), Tdt | |
| | | | (2+, S), CD1a (1+, S), CD34 | |
| | | | (−), Fli-1 (3+, W), CD7 (4+, | |
| | | | S) | |
| CRL-2264 | CEM/C2 | T-cell leukemia | Nothing stained | |
| CRL-2294 | BCP-1 | Body cavity lymphoma | HHV8 (3+, S) | CD3(−), CD20 (−), CD5 (−), |
| | | | | CD79a (−), Bcl2 (−), Bcl6 (−), |
| | | | | Tdt (−), CD10 (−) EBV (in |
| | | | | situ) (−) |
| CRL-2505 | 22RV1 | Prostatic carcinoma | AR (3+, I), MOC31 (4+, S), | PSA (−), P504S (−), PAP (−), |
| | | | BerEP4 (4+, S) | MUC1 (−), |
| PCS-440-010 | N/A | Normal prostate | P504S (3+) | PSA (−), PAP (−), NKX3.1 (−), |
| | | | | ERG (−), AR (−), BK virus (−) |
| CRL-2551 | Panc8.13 | Pancreatic | Maspin (4+, S), S100P (3+, | pVHL (−), p53 (−), CK17 (−) |
| | | adenocarcinoma | S), KOC (3+, S), MUC5AC | |
| | | | (3+, S), CEA (4+, S) | |
| CRL-2547 | Panc10.05 | Pancreatic | Maspin (2+, I), KOC (4+, S), | MUC5AC (−), CK17 (−), CEA |
| | | adenocarcinoma | S100P (2+, W), p53 (3+, S) | (−) |
| CRL-2549 | Panc3.27 | Pancreatic | Maspin (4+, S), MUC5AC | p53 (−), CEA (−) |
| | | adenocarcinoma | (3+, S), KOC (3+, I), S100P | |
| | | | (3+, S), pVHL (1+, W), CK17 | |
| | | | (1+, S) | |
| CRL-185 | A549 | Lung adenocarcinoma | CK7 (2+, I) | TTF-1 (−), Napsin A (−), |
| | | | | MOC31 (−), BerEP4 (−), KOC |
| | | | | (−), MUC1 (−), p63 (−), |
| | | | | CK5/6 (−) |
| CRL-5895 | NCI-H1792 | Lung Adenocarcinoma | CK7 (4+, S) KOC (3+W), | TTF-1 (−), Napsin A (−), |
| | | | MUC1 (1+, W), p63 (1+, I) | MOC31 (−), BerEP4 (−), CEA |
| | | | | (−), CK5/6 (−) |
| CRL-5826 | NCI-H226 | Lung Adenocarcinoma | CK7 (4+), KOC (4+, S) | TTF-1 (−), Napsin A (−), |
| | | | | S100 (−), CEA (−), ALK(−) |
| HTB-184 | NCI-H510A | Lung Adenocarcinoma | CK7 (3+, I), TTF1 (4+, S), | Napsin A (−), S100 (−), |
| | | | CEA (4+, S), MOC31 (4+, S) | |
| CRL-5908 | NCI-H1975 | Lung Adenocarcinoma | TTF1 (1+, S), CK7 (4+, S), | CEA (−), Napsin A (−) |
| | | | MOC31 (3+, S) | |
| CRL-5877 | NCI-H1573 | Lung adenocarcinoma | BerEP4 (4+, S), MOC31 | TTF1 (−), napsin A (−) |
| | | | (4+, S), CEA (4+, S) | |
| CCL-256 | NCI-H-2126 | Lung adenocarcinoma | MOC31 (4+, S), BerEP4 | TTF1(−), Napsin A (−) |
| | | | (4+, S), CEA (4+, S) | |
| CRL-5869 | NCI-H1417 | Lung, small cell carcinoma | TTF1 (3+, W), CD56 (4+, S), | p53 (−), CK7 (−) |
| | | | synaptophysin (4+, S), | |
| | | | chromogranin (3+, S), Ki67 | |
| | | | (70%), NSE (3+, W), | |
| | | | CAM5.2 (3+, I) | |
| CCL-257 | NCI-H1688 | Lung, small cell carcinoma | TTF1 (2+, I), CD56 (4+, S), | NSE (−), p53 (−), |
| | | | synaptophysin (4+, S), | |
| | | | chromogranin (3+, S), | |
| | | | Ki67 >80%, CK7 (4+, S), | |
| | | | CAM5.2 (3+, I) | |
| CRL-2279 | | Endothelial cells | ERG (4+, S), Fli-1 (4+, S), BK | CD31 (−), CD34 (−), Factor 8 |
| | | | virus (4+, S) | (−), TBM (−), vimentin (−) |
| CRL-5946 | NCI-H2452 | Mesothelioma | CK7 (4+, S), D2-40 (2+, I), | CK5/6 (−) |
| | | | calretinin (1+, S), CK5/6 (−), | |
| | | | WT-1 (2+, W), HBME1 (1+, | |
| | | | W), KOC (2+, W), TBM (2+, | |
| | | | S) | |
| CRL-2234 | SUN-449 | Hepatocellular carcinoma | CD10 (4+, S) | MOC31 (−), glypican3 (−), |
| | | | | HepPr1 (−), Arg1 (−), B- |
| | | | | catenin (4+, M), HepBsAg |
| | | | | (−), HepBcAg (−) |
| CRL-2237 | SUN-387 | Hepatocellular carcinoma | CD10 (4+, S) | MOC31 (−), glypican3 (−), |
| | | | | HepPr1 (−), Arg1 (−), B- |
| | | | | catenin (4+, M), HepBsAg |
| | | | | (−), HepBcAg (−) |
| CRL-11233 | THLE3 | Normal liver +SV40 | CD10 (4+, S), BK (4+, S)) | Arg-1 (−), AFP (−), |
| | | | | HepPar1(−), glypican 3 (−), |
| | | | | pCEA (−) |
| CRL-2706 | THLE2 | Normal liver +SV40 | CD10 (4+, S), BK (4+, S) | Arg-1 (−), AFP (−), |
| | | | | HepPar1(−), glypican 3 (−), |
| | | | | pCEA (−) |
| CRL-10741 | C3A | Hepatocellular carcinoma | AFP (4+, S), glypican-3 (4+, | Arg-1 (−), HepPar1 (−), |
| | | | S), MOC31 (4+, S), CAM5.2 | CD10 (−), B-catenin (M, +) |
| | | | (4+, S) | |
| HB-8065 | HepG2 | Hepatocellular carcinoma | AFP (4+, S), glypican-3 (4+, | Arg-1 (−), HepPar1 (−), |
| | | | S), MOC31 (4+, S), CAM5.2 | CD10 (−), B-catenin (M, +) |
| | | | (4+, S) | |
| CRL-1830 | Hepal-6 | Hepatocellular carcinoma | B-catenin (M+) | Arg 1 (−), Hepar1 (−), |
| | | | | glypican 3 (−), CD10 (−), |
| | | | | CEA-P (−), AFP (−), CAM5.2 |
| | | | | (−), CD31 (−), CD34 (−) |
| CRL-2279 | MS1 | Islet cells (pancreas) | | Synaptophysin (−), |
| | | | | chromogranin (−), insulin (−), |
| | | | | glucogan (−) |
| CRL-1803 | TT | Thyroid, medullary | Calcitonin (3+, S), CEA (4+, | HBME1 (−), galectin3 (−), |
| | | carcinoma | S), synaptophysin (4+, S), | CK19 (−), S100A1 (−), |
| | | | chromogranin (3+, S), | thyroglobulin (−) |
| | | | CD56 (4+, S), TTF1 (4+, S), | |
| | | | S100A6 (4+, S), vimentin | |
| | | | (1+), PAX8 (2+, S), cyclin | |
| | | | D1 (4+, S), | |
| HTB-36 | JEG-3 | Placenta, choriocarcinoma | SALL4 (4+, S), S100P (2+, | p57 (−), OCT4 (−), SOX2 (−), |
| | | | S), PLAP (2+, S), HCG (2+, | Nanog (−) |
| | | | S), CD10 (4+, S) | |
| CRL-2180 | L2-RYC | Yolk sac tumor | SALL4 (4+, S), CD117 (2+, I, | glypican3 (−), HepPar1 (−), |
| | | | but nuclear stain), AFP- | OCT4 (−), PLAP (−), Nanog (−), |
| | | | not done yet | CD30 (−), D2-40 (−) |
| HTB-105 | Tara-1 | Embryonal carcinoma | SALL4 (4+, S), OCT4 (4+, S), | CD117 (−), HCG (−), |
| | | | PLAP (4+, S), AFP (1+, S), | glypican 3 (−) |
| | | | CD30 (1+, S), Nanog (4+, S) | |
| CRL-2073 | NCCIT | Teratocarcinoma | SALL4 (4+, S), OCT4 (4+, S), | Beta-HCG (−) |
| | | | glypican 3 (2+, S), D2-40 | |
| | | | (3+, S), PLAP (1+, S), CD30 | |
| | | | (3+, S), AFP (1+, S), Nanog | |
| | | | (4+, S) | |
| CCL-136 | RD | Rhabdomyosarcoma | Desmin (4+, S), MyoD1 | MSA (−) |
| | | | (4+, S), myogenin (3+, S), | |
| | | | SMA (3+, I) | |
| CRL-2946 | UWB1.289 + BRCA1 | Ovarian carcinoma | PAX8 (4+, S), PAX2 (1+, W) | ER (−), PR (−), WT1 (−), |
| | | | | HNF1B (−), p53 (−), CDX2 (−) |
| HTB35 | SiHa | Cervical carcinoma | P16 (4+, S), CK7 (4+, S), | CK5/6 (−), CK903 (−), p63 (−), |
| | | | HPV (in situ) (1+), S100P | p40 (−), CEA (−), ER (−), PR |
| | | | (1+, S), CK5/6 (−), CK903 (−), | (−) |
| | | | GATA3 (1+), p63 (−), p40 | |
| | | | (−), CEA (−), ER (−), PR (−) | |
| CRL-1550 | Ca-ski | Cervical Carcinoma | P16 (4+, S), CK7 (4+, S), | CEA (−), ER (−), PR (−), |
| | | | HPV (in situ) (4+, S)), CK5/6 | GATA3 (−) |
| | | | (4+, S), CK903 (4+, S), p63 | |
| | | | (4+, S), p40 (4+, S) | |
| CRL-11882 | Hs-5 | Stromal cells (HPV+) | P16 (4+, S), vimentin (4+, | |
| | | | S), HPV (in situ) (1+, W) | |
| CRL-10296 | NCI-H295 | Adrenal cortical carcinoma | Synaptophysin (3+, S), | Mart-1 (−), chromogranin |
| | | | vimentin (4+, S) | (−), inhibin-a (−), calretinin |
| | | | | (−), SF-1 (−) |
| HTB166 | RD-ES | Ewing sarcoma | CD99 (4+, S), NKX2.2 (4+, | NSE (−), WT-1 (−), desmin (−) |
| | | | S), Fli-1 (2+, W), Vimentin | |
| | | | (2+, S), CD56 (1+, S) | |
| CRL-7556 | Hs-822.T | Ewing sarcoma | CD99 (4+, S), Fli-1 (2+, I), | NKX2.2 (−), NSE (−), WT-1 (−), |
| | | | vimentin (4+, S), CD56 (2+, | desmin (−) |
| | | | N+?) | |

TABLE 4
Summary of specific cell lines used to construct a set of TMA control blocks for selected biomarkers

| | | | | | Targeted |
|---|---|---|---|---|---|
| | | | | Targeted Biomarkers | Biomarkers with |
| Names of | Ratio of Each | Name of Control | Types of Cancer to Be | with high level | low level and no |
| Cell Lines from ATCC | Cell Line | Block | Covered | expression | expressions |
| HTB-133 | HTB-133 - 40% | Breast Cancer | Breast ductal and lobular | CK7, ER, PR, HER2, | ER, PR, HER2, |
| CRL-2330 | CRL-2330 - 40% | | carcinomas | GATA3, GCDFP15, | GATA3, GCDFP15, |
| CCL-253 | CCL-253 - 20% | | | MGB, TFF3, TFF1 | MGB, TFF3, TFF1 |
| G361 | G361 - 40% | Melanoma | Melanocytic tumors | S100 , HMB45, | S100, HMB45, |
| CRL-1585 | CRL-1585 - 40% | | | SOX10, Mart-1, MiTF, | SOX10, Mart-1, |
| CRL-5895 | CRL-5895 - 20% | | | Vimentin, SOX2, | MiTF, Vimentin, |
| | | | | S100A6, MUM1, | SOX2, S100A6, |
| | | | | AE1/3, CK7 | MUM1, AE1/3, CK7 |
| CRL-1582 | CRL-1582 - | Lymphoma | B-cell and T-cell | CD2, CD3, CD5, | CD2, CD3, CD5, |
| CCL-86 | 40% | | lymphomas | CD20, CD79a, PAX5, | CD20, CD79a, |
| CRL-5895 | CCL-86 - 40% | | | CD10, Tdt, EBV, Bcl2, | PAX5, CD10, Tdt, |
| | CRL-5895 - 20% | | | Bcl6, CK and CK7 | EBV, Bcl2, Bcl6, CK |
| | | | | | and CK7 |
| CRL-2073 | CRL-2073 - | Germ Cell Tumor | Seminoma, | SALL4, S100P, PLAP, | SALL4, S100P, |
| HTB-36 | 25% | | Embryonal carcinoma, | Beta-HCG, CD10, | PLAP, Beta-HCG, |
| HepG2 (BB- | HTB-36 - 25% | | Yolk sac tumor, | OCT4, AFP, glypican- | CD10, OCT4, AFP, |
| 8065) | HepG2 (BB- | | Choriocarcinoma, | 3, D2-40, CD30, | glypican-3, D2-40, |
| CRL-1585 | 8065) - 25% | | Teratoma | Nanog, SOX2, | CD30, Nanog, |
| | CRL-1585 - 25% | | | Cytokeratin | SOX2, Cytokeratin |
| CCL-136 | CCL-136 - | Malignant Small | Lymphoma/leukemia, Small | Desmin, MyoD1, | Desmin, MyoD1, |
| HTB166 | 20% | Round Cell Tumor | cell carcinoma/ | myogenin, Smooth | myogenin, Smooth |
| TT (CRL-1803) | HTB166 - 20% | | neuroendocrine carcinoma, | muscle actin, CD99, | muscle actin, CD99, |
| CRL-1582 | TT (CRL- | | Ewing's sarcoma/PNET, | NKX2.2, Fli-1, | NKX2.2, Fli-1, WT- |
| CCL-86 | 1803) - 20% | | Rhabdomyosarcoma, | synaptophysin, | 1, synaptophysin, |
| CRL-5946 | CRL-1582 - | | Neuroblastoma, | chromogranin, CD56, | chromogranin, |
| | 20% | | Leiomyosarcoma, | WT-1, NSE, vimentin, | CD56, NSE, |
| | CCL-86 - 20% | | Desmoplastic small round | TTF1, Cytokeratin, Tdt, | vimentin, TTF1, |
| | CRL-5946 - 20% | | cell tumor | CD3, CD20, EBV | Cytokeratin, Tdt, |
| | | | | | CD3, CD20, EBV |
| HTB133 | HTB133 - 10% | Tumor of Known | To Include a tumor from the | ER, PR, GATA3, | ER, PR, GATA3, |
| CCL-253 (NCI- | CCL-253 (NCI- | Primary | lung, breast, pancreas, | GCDFP15, CK7, | GCDFP15, CK7, |
| H508) | H508) - 10% | | kidney, thyroid, uterus, | CK20, CK5/6, CK903, | CK20, CK5/6, |
| TT (CRL-1803) | TT (CRL-1803) - | | uterine cervix, ovary, upper | p40, p63, CDX2, CEA, | CK903, p40, p63, |
| Panc 3.27 (CRL- | 10% | | GI tract, colon, bladder, | MOC31, B72.3, | CDX2, CEA, |
| 2549) | Panc 3.27 (CRL- | | prostate, vascular tumors | BerEP4, Beta-catenin, | MOC31, B72.3, |
| CRL-1932 | 2549) - 10% | | and neuroendocrine tumors | SATB2, CDH17, | BerEP4, Beta- |
| CRL-2279 | CRL-1932 - 10% | | | TTF1, calcitonin, | catenin, SATB2, |
| CRL-1550 | CRL-2279 - 10% | | | chromoganin, | CDH17, TTF1, |
| CRL-5946 | CRL-1550 - 10% | | | synaptophysin, CD56, | calcitonin, |
| | CRL-5946 - 10% | | | Maspin, MUC1, | chromogranin, |
| | | | | MUC2, MUC5AC, | synaptophysin, |
| | | | | IMP3, S100P, PAX8, | Maspin, MUC1, |
| | | | | PAX2, vimentin, | MUC2, MUC5AC, |
| | | | | P504S, ERG, Fli-1, | IMP3, S100P, PAX8, |
| | | | | p16, WT-1, HPV (in | PAX2, vimentin, |
| | | | | situ) | P504S, ERG, Fli-1, |
| | | | | | p16, HPV (in situ) |
| HTB166 | HTB166 - 20% | Sarcoma | Leiomyosarcoma | Desmin, myoD1, | Desmin, myoD1, |
| CCL-136 | CCL-136 - 20% | | Rhabdomyosarcoma | myogenin, SMA, | myogenin, SMA, |
| CRL-2279 | CRL-2279 - 20% | | Ewing sarcoma | CD99, NKX2.2, Fli-1, | CD99, NKX2.2, Fli- |
| CRL1585 | CRL1585 - 20% | | Neurogenic tumors | ERG, vimentin, S100, | 1, ERG, vimentin, |
| CRL-1550 | CRL-1550 - 20% | | Vascular tumors | cytokeratin, CK5/6, | S100, cytokeratin, |
| | | | Sarcomatoid carcinomas | CK903, p63, p40, p16 | CK5/6, CK903, p63, |
| | | | | | p16 |

TABLE 5
Tested antibodies and staining protocols

| | | | | | | | Control |
|---|---|---|---|---|---|---|---|
| Antibody | Cat No. | Clonality | Host Animal | Dilution | Retrieval | Incubation | Tissue |
| Actin, Muscle | Ventana 760-2601 | HHF35 | Mouse | Predilute | None | 16 min. | Soft tissue |
| Specific (MSA) | | | | | | | tumors with |
| | | | | | | | muscle |
| Actin, Smooth | DAKO. M0851 | 1A4 | Mouse | 1:150 | CC1 short | 8 min. @ 37° C. | Leiomyosarcoma |
| Muscle (SMA) | | | | | | | |
| Alpha- | DAKO. N1501 | Polyclonal | Rabbit | 1:2 | CC1 standard | 40 min. @ 37° C. | Germ Cell Tumor |
| Fetoprotein (AFP) | | | | | | | |
| Alpha-methylacyl- | BioM PP365JJ | Polyclonal | Rabbit | Predilute | CC1 mild | 16 min. @ 37° C. | Prostate |
| CoA racemase | | | | | | | |
| AMACR (P504S) | | | | | | | |
| Androgen | CellMq.200M-18 | AR441 | Mouse | Predilute | CC1 | 36 min. @ 37° C. | Prostate |
| Receptor Protein | | | | | standard | | |
| (ARP) | | | | | | | |
| Arginase-1 | Epitomics | EPR6672(B) | Rabbit | 1:500 | CC1 mild | 32 min. @ 37° C. | Normal liver |
| | 5222-1 | | | | | | |
| B-cell | Ventana 760-4240 | 124 | Mouse | Predilute | CC1 standard | 16 min. heat | Tonsil |
| CLL/lymphoma 2 | | | | | | disabled | |
| Bcl-2 | | | | | | | |
| B-cell | Ventana 760-4241 | GI191E/A8 | Mouse | Predilute | CC1 standard | 32 min. @ 37° C. | Tonsil |
| CLL/lymphoma 6 | | | | | | | |
| Bcl-6 | | | | | | | |
| Bcl-1 (cyclin D1) | Ventana 760-4282 | SP4 | Rabbit | Predilute | CC1 standard | 32 min. @ 37° C. | Mantle cell |
| | | | | | | | lymphoma |
| Ber-EP4 | DAKO. M0804 | Ber-EP4 | Mouse | 1:100 | Protease 1 | 32 min. @ 37° C. | #2 |
| | | | | | 4 min. | | |
| Beta-Catenin | Ventana 760-4242 | 14 | Mouse | Predilute | CC1 mild | 16 min. heat | #2 |
| | | | | | | disabled | |
| BRST-2 (GCDFP- | CellMq .257M | 23A3 | Mouse | Predilute | CC1 standard | 32 min.@ 37° C. | Breast CA |
| 15) | | | | | | | |
| BRST-3 (TAG 72) | Ventana 760-2669 | B72.3 | Mouse | Predilute | CC1 mild | 24 min. @ 37° C. | #2 |
| BSAP (PAX5) | Ventana 760-4270 | 24 | Mouse | Predilute | CC1 standard | 32 min. @ 37° C. | Tonsil |
| Cadherin-17 | Epitomics | EP86 | Rabbit | 1:50 | CC1 mild | 32 min. @ 36° C. | Colon ADC |
| | AC-0095 | | | | | | |
| | RUO | | | | | | |
| Calcitonin | Ventana 760-2611 | Polyclonal | Rabbit | Predilute | CC1 mild | 32 min.@ 37° C. | Medullary |
| | | | | | | | thyroid CA |
| Caldesmon | Ventana 760-4375 | E89 | Rabbit | Predilute | CC1 standard | 32 min.@ 37° C. | Smooth muscle |
| Calponin-1 (CALP- | Ventana 760-4376 | EP798Y | Rabbit | Predilute | CC1 standard | 32 min.@ 37° C. | Uterus |
| 1) | | | | | | | |
| Calretinin | Ventana 760-2700 | Polyclonal | Rabbit | Predilute | CC1 mild | 32 min.@ 37° C. | Mesothelial cells |
| (CALRET) | | | | | | | |
| Carbonic | CellMq | MRQ-54 | Mouse | 1:50 | CC1 mild | 32 min. @ 36° C. | Clear cell RCC |
| anhydrase IX (CA | 379M | | | | | | |
| IX) | | | | | | | |
| Carcinoembryonic | Biogx MU009-UC | B01-94-11M-P | Mouse | 1:250 | CC1 short | 40 min. | #2 |
| Antigen (CEA) | | | | | | | |
| CD10 | Ventana 760-2705 | 56C6 | Mouse | Predilute | CC1 mild | 56 min.@ 42° C. | Tonsil |
| CD117 (c-kit) | Ventana 790-2951 | 9.7 | Rabbit | Predilute | CC1 mild | 20 min. | CD117 (GIST) |
| CD138 | Ventana 760-4248 | B-A38 | Mouse | Predilute | CC1 mild | 28 min. @ | Tonsil |
| | | | | | | 37° C. | |
| CD15 | Ventana 760-2504 | MMA | Mouse | Prediluted | CC1 mild | 40 min. | Tonsil |
| CD19 | DAKO. M7296 | LE-CD19 | Mouse | 1:50 | CC1 standard | 40 min.@ 37° C. | Tonsil |
| CD1a | Ventana 760-4244 | MTB1 | Mouse | Predilute | CC1 mild | 40 min. @ 37° C. | Skin |
| CD2 | Biogx MU438UC | AB75 | Mouse | 1:30 | CC1 mild | 32 min.@ 37° C. | Tonsil |
| CD20 | Ventana 760-2531 | L26 | Mouse | Predilute | CC1 short | 16 min. | Tonsil |
| CD3 | Ventana 760-4341 | 2GV6 | Rabbit | Predilute | CC1 standard | 32 min.@ 37° C. | Tonsil |
| CD30 (Ber H-1) | Ventana 790-2926 | BerH2 | Mouse | Predilute | CC1 standard | 20 min. | Embryonal CA |
| CD31 | DAKO M0823 | JC7OA | Mouse | 1:30 | Protease 1 | 32 min.@ 37° C. | Tonsil |
| | | | | | 8 min. | | |
| CD34 | Ventana 790-2927 | QBEnd/10 | Mouse | Predilute | No retrieval | 8 min. | Tonsil |
| CD43 | Ventana 7600-2511 | L60 | Mouse | Predilute | CC1 mild | 20 min. | Tonsil |
| CD44 | DAKO.M7082 | DF1485 | Mouse | 1:80 | CC1 standard | 40 min.@ 37° C. | Tonsil |
| CD45 (LCA) | DAKO. M0701 | 2B11 + PD7/26 | Mouse | 1:1600 | CC1 mild | 28 min. | Tonsil |
| CD5 | VECTOR VP-C322 | 4C7 | Mouse | 1:400 | CC1 Mild | 28 min.@ 37° C. | Tonsil |
| CD56 | Ventana 760-2625 | 123C3.D5 | Mouse | Predilute | CC1 mild | 40 min. | Neuroblastoma |
| | | | | | | | #4 |
| CD57 | Ventana 760-2626 | NK1 | Mouse | Predilute | CC1 standard | 20 min. @37° C. | Tonsil |
| CD79a | Ventana 790-2932 | 11E3 | Mouse | Predilute | CC1 mild | 12 min. | Tonsil |
| CD8 | Ventana 760-4250 | C8/144B | Mouse | Predilute | CC1 standard | 8 min.@ 37° C. | Tonsil |
| CD99 | Ventana 760-2631 | H036-1.1 | Mouse | Predilute | CC1 mild | 16 min @ 37° C. | Ewing's Sarcoma |
| CDX-2 | Biogx MU392A-UC | CDX2-88 | Mouse | 1:30 | CC1 mild | 32 min. | Colon CA |
| Chromogranin A | BiocareM PM010- | LK2H10 + PHE5 | Mouse | Predilute | CC1 standard | 32 min. @37° C. | Pancreas |
| | AA | | | | | | |
| Cyclin D1 | Ventana 760-4282 | SP4 | Rabbit | Predilute | CC1 standard | 32 min. @ 37° C. | Mantle cell |
| | | | | | | | lymphoma |
| Cytokeratin | Ventana 790-4373 | 34betaE12 | Mouse | Predilute | Protease 1 | 24 min. @ 37° C. | prostate |
| (34betaE12) | | | | | 4 min. | | |
| Cytokeratin | CellMq .335M-96 | 35betaH11 | Mouse | 1:100 | CC1 mild | 32 min. @ 37° C. | prostate |
| (35betaH11) CK8 | | | | | | | |
| Cytokeratin 14 | CellMq.314M-16 | LL002 | Mouse | 1:200 | CC1 mild | 32 min.@ 37° C. | #2 |
| (CK14) | | | | | | | |
| Cytokeratin 17 | DAKO.M7046 | E3 (1). | Mouse | 1:80 | CC1 standard | 32 min.@ 37° C. | #2 |
| (CK17) | | | | | | | |
| Cytokeratin 19 | Ventana 760-4281 | (A53-B/A2.26) | Mouse | Predilute | CC1 short | 20 min.@ 37° C. | #2 |
| (CK19) | | | | | | | |
| Cytokeratin 20 | Ventana 760-2635 | Ks20.8 | Mouse | Predilute | CC1 mild | 28 min., no heat | #2 |
| (CK20) | | | | | | | |
| Cytokeratin 5 & 6 | Ventana 760-4253 | D5 &16B4 | Mouse | Predilute | CC1 mild | 32 min.@ 37° C. | Prostate |
| (CK5/6) | | | | | | | |
| Cytokeratin 5 | CellMq .305R15 | EP1601Y | Rabbit | 1:50 | CC1 standard | 32 min.@ 37° C. | mesothelioma |
| (CK5) | | | | | | | |
| Cytokeratin 7 | CellMq. 307M-95 | OV-TL12/30 | Mouse | 1:200 | CC1 mild | 32 min.@ 37° C. | #2 |
| (CK7) | | | | | | | |
| Cytokeratin 8 & | CellMq.818M | B22.1&B23.1 | Mouse | Predilute | CC1 standard | 32 min. @ 37° C. | Pancreas, |
| 18 (CK8/18) | | | | | | | prostate, salivary |
| | | | | | | | gland |
| Cytokeratin | DAKO. M3515 | AE1/AE3 | Mouse | 1:100 | Protease 1 | 32 min. @ 37° C. | #2 |
| Cocktail | | | | | 4 min. | | |
| (AE1/AE3) | | | | | | | |
| Cytokeratin, | Ventana 790-4373 | 34betaE12 | Mouse | Predilute | Protease 1 | 24 min. @ 37° C. | Prostate |
| HMW/CK903 | | | | | 4 min. | | |
| Cytokeratin, | Becton Dickinson. | CAM5.2 | Mouse | 1:4 | CC1 mild | 20 min. @ 37° C. | #2 |
| LMW/CAM 5.2 | 349205 | | | | | | |
| D2-40 | DAKO. M3619 | D2-40 | Mouse | 1:50 | CC1 short | 32 min. @ 37° C. | Tonsil |
| (Podoplanin) | | | | | | | |
| Desmin | VBS ORG-8889 | DE-R-11 | Mouse | Predilute | CC1 standard | 28 min., no heat | #2 |
| E-Cadherin | DAKO.M3612 | NCH-384 | Mouse | 1:200 | CC1 mild | 48 min. | #2 |
| Epithelial | Ventana 760-4259 | E29 | Mouse | Predilute | CC1 mild | 16 min. @ 37° C. | #2 |
| Membrane | | | | | | | |
| Antigen (EMA) | | | | | | | |
| ERG | BiocareM | 9FY | Mouse | Predilute | CC1 extended | 36 min. | Vascular tumors |
| | VP421G5 | | | | | | |
| Estrogen | Ventana 790-4324 | SP1 | Rabbit | Predilute | CC1 mild | 40 min. @ 37° C. | ER |
| Receptor (ER) | | | | | | | |
| FLI-1 | CellMq. 254M-15 | MRQ-1 | Mouse | 1:50 | CC1 standard | 32 min @37° C. | angiosarcoma |
| Galectin-3 (GAL- | Ventana 760-4256 | 9C4 | Mouse | Predilute | CC1 mild | 24 min. @ 37° C. | Pancreas CA |
| 3) | | | | | | | |
| GATA-3 | BiocareM | L50-823 | Mouse | 1:400 | CC1 standard | 32 min. @ 37° C. | Breast carcinoma |
| | CM405 | | | | | | |
| Glial Fibrillary | DAKO.M0761 | 6F2 | Mouse | 1:150 | CC1 mild | 32 min. @ 37° C. | Brain |
| Acidic Protein | | | | | | | |
| (GFAP) | | | | | | | |
| Glypican-3 (GPC3) | CellMq.261M-98 | 1G12 | Mouse | Predilute | CC1 mild | 32 min. @ 37° C. | Liver CA |
| Gross Cystic | CellMq.257M-18 | 23A3 | Mouse | Predilute | CC1 standard | 32 min. @ 37° C. | Breast CA |
| Disease Fluid | | | | | | | |
| Protein 15 | | | | | | | |
| (GCDFP-15) | | | | | | | |
| HBME-1 | CellMq.283M-18 | HBME-1 | Mouse | Predilute | CC1 mild | 32 min. @ 37° C. | Mesothelioma |
| (Mesothelial Cell) | | | | | | | |
| HepPar1 | DAKO. M7158 | OCH1E5 | Mouse | 1:80 | CC1 mild | 20 min. | Liver |
| Her-2/neu (c- | Ventana 790-100 | 4B5 | Rabbit | Predilute | CC1 mild | 16 min. no heat | Breast |
| erbB-2, Her-2) | | | | | | | |
| HNF-1beta | Sigma | Polyclonal | Rabbit | 1:100 | CC1 standard | 32 min. @ 37° C. | Clear cell |
| | HPA002083 | | | | | | carcinoma of the |
| | | | | | | | ovary |
| Human Chorionic | DAKO. A0231 | Polyclonal | Rabbit | 1:2000 | CC1 mild | 32 min. @ 37° C. | Placenta |
| Gonadotropin | | | | | | | |
| (hCG) | | | | | | | |
| Human Herpes | Ventana 760-4260 | 13B10 | Mouse | Predilute | CC1 mild | 32 min. @ 37° C. | Kaposi's Sarcoma |
| Virus 8 (HHV-8) | | | | | | | |
| Human Melanoa | CellMq.282M-98 | HMB45¹ | Mouse | Predilute | CC1 standard | 24 min. @ 37° C. | Melanoma |
| Black-45 (HMB- | | | | | | | |
| 45) | | | | | | | |
| Human Placental | Ventana 760-4443 | Polyclonal | Rabbit | Predilute | CC1 mild | 40 min. @ 37° C. | Placenta |
| Lactogen (hPL) | | | | | | | |
| Inhibin, alpha | Ventana 760-2634 | R1 | Mouse | Predilute | CC1 mild | 24 min. @ 37° C. | Adrenal gland |
| (IHN-A) | | | | | | | |
| INI-1, hSNF5 (INI- | CellMq.272M-16 | MRQ-27 | Mouse | 1:30 | CC1 standard | 24 min. @ 37° C. | Brain |
| I) | | | | | | | |
| Kappa | Ventana 760-2514 | L1C1 | Mouse | Predilute | Protease 1 | 32 min. @ 37° C. | Tonsil |
| | | | | | 4 min. | | |
| Ki-67 | Ventana 790-4286 | K2 | Mouse | Predilute | CC1 mild | 16 min. | Tonsil |
| KOC (IMP-3) | DAKO.M3626 | 69.1 | Mouse | 1:100 | CC1 extended | 32 min. @ 37° C. | Lung/ |
| | | | | | | | Pancreatic CA |
| Lamba Light | Ventana 760-2515 | Polyclonal | Rabbit | Predilute | CC1 standard | 8 min. | Tonsil |
| Chains (Lambda) | | | | | | | |
| Mammaglobin | Ventana 760-4263 | 31-A5 | Mouse | Predilute | CC1 standard | 40 min. @ 42° C. | Breast |
| (MGB) | | | | | | | CA(Ductal) |
| MART-1/Melan A | Ventana 790-2990 | A103 | Mouse | Predilute | CC1 mild | 16 min. @ 37° C. | Melanoma |
| Maspin | BD. 554292 | G167-70 | Mouse | 1:200 | CC1 mild | 40 min. @ 37° C. | Pancreatic CA |
| Melan-A | DAKO. M7196 | A103 | Mouse | 1:30 | CC1 mild | 32 min. @ 37° C. | Skin |
| Microphthalmia | DAKO.M3621 | D5 | Mouse | 1:80 | CC1 mild | 32 min. @ 37° C. | Melanoma |
| Transcription | | | | | | | |
| Factor (MiTF) | | | | | | | |
| MLH1 | Ventana 760-4264 | G168/728 | Mouse | Predilute | CC1 standard | 32 min. @ 37° C. | #2 |
| MOC-31 | DAKO.M3525 | MOC-31 | Mouse | 1:50 | CC1 mild | 32 min. @ 37° C. | #2 |
| MSH2 | Ventana 760-4265 | G219-1129 | Mouse | Predilute | CC1 mild | 32 min. | #2 |
| MSH6 | Zymed 610918 | 44/MSH6 | Mouse | 1:50 | CC1 standard | 48 min. @ 37° C. | #2 |
| MUC1 | VECTOR.VP-M654 | Ma 552 | Mouse | 1:200 | CC1 standard | 40 min. @ 37° C. | Breast CA |
| MUC2 | VECTOR.VP-M656 | Ccp 58 | Mouse | 1:100 | CC1 standard | 40 min. @ 37° C. | Small bowel |
| MUC4 | BiocareM CM326 | 8G-7 | Mouse | 1:200 | CC1 mild | 40 min. @ 37° C. | #2 |
| MUC5AC | VEC.VP-M657 | CLH2 | Mouse | 1:50 | CC1 standard | 40 min. @ 37° C. | Stomach |
| MUC6 | VEC.VP-M658 | CLH5 | Mouse | 1:50 | CC1 standard | 40 min. @ 37° C. | Stomach |
| Myeloperoxidase | DAKO. A0398 | Polyclonal | Rabbit | 1:2400 | CC1 mild | 32 min. @ 37° C. | Myeloid |
| (MPO) | | | | | | | leukanemia |
| Nanog | Abcam | EPR2027(2) | Rabbit | 1:100 | CC1 standard | 32 min. @ 36° C. | Seminoma |
| | Ab109250 | | | | | | |
| Napsin A | Ventana 760-4446 | polyclonal | Rabbit | Predilute | CC1 mild | 40 min. No heat | LungCA kidney |
| Neuron-Specific | Ventana 760-2662 | E27 | Mouse | Predilute | CC1 mild | 12 min. no heat | Appendix |
| Enolase (NSE) | | | | | | | |
| NKX2.2 | Columbia U. | 74.5A5 | Mouse | 1:100 | CC1 mild | 32 min. @ 36° C. | PNET/Ewing's |
| | 74.5A5 | | | | | | Sarcoma |
| | DSHB@UIOWA.EDU | | | | | | |
| NKX3.1 | BiocareM | Polyclonal | Rabbit | 1:50 | CC1 standard | 32 min. @ 36° C. | Prostatic |
| | CP422B | | | | 100° C. | | carcinoma |
| OCT4 | CellMq. CXA122 | MRQ-10 | Mouse | Predilute | CC1 mild | 32 min. @ 37° C. | Seminoma |
| p504S/AMACR | BiocareM PP365JJ | Polyclonal | Rabbit | Predilute | CC1 mild | 16 min. @ 37° C. | Prostate |
| p53 | Ventana 790-2912 | D0-7 | Mouse | Predilute | CC1 mild | 20 min. @ 37° C. | #2 |
| p63 | BiocareM | BC4A4 | Mouse | Predilute | CC1 48 min. | 24 min. @ 36° C. | Breast /Prostate |
| | VP163G | | | | 100° C. | | |
| Paired Box Gene- | CellMq. 311A-18 | Polyclonal | Rabbit | Predilute | CC1 standard | 24 min. Heat | Renal CA |
| 2 (PAX2) | | | | | | disabled | |
| Pancytokeratin | DAKO. M0821 | MNF116 | Mouse | 1:100 | CC1 mild | 20 min.@ 37° C. | Epithelial tumors |
| (pan-CK) | | | | | | | |
| PAX5 | Ventana | SP34 | Rabbit | Predilute | CC1 standard | 32 min. @ 36° C. | Tonsil |
| | 790-4420 | | | | | | |
| PAX8 | BiocareM. CP379AK | N/A | Rabbit | 1:20 | CC1 standard | 32 min. @37° C. | Renal tissue |
| Phospho-histone | Cellsig #9714 | Polyclonal | Rabbit | 1:50 | CC1 standard | 40 min. @37° C. | Tonsil |
| H3 (PHH3) | | | | | | | |
| Placental Alkaline | Ventana 760-2664 | NB10 | Mouse | Predilute | CC1 mild | 32 min..@ 37° C. | Placenta |
| Phosphatase | | | | | | | |
| (PLAP) | | | | | | | |
| PMS2 | BD Parmingen | A16-4 | Mouse | 1:30 | CC1 standard | 48 min.@ 37° C. | Colon CA |
| | 556415 | | | | | | |
| Progesterone | Ventana 790-2223 | 1E2 | Rabbit | Predilute | CC1 mild | 24 min. no heat | Breast CA |
| Receptor (PR) | | | | | | | |
| PSA (prostate | | | | | | | |
| specific antigen) | | | | | | | |
| Renal cell | Ventana 760-4273 | PN-15 | Mouse | Predilute | Protease 1 | 40 min.@ 37° C. | RCC |
| carcinoma marker | | | | | 8 min. | | |
| (RCCMa) | | | | | | | |
| S100 | Ventana 790-2914 | 4C4.9 | Mouse | Predilute | CC1 mild | 8 min.Heat | Melanoma |
| | | | | | | disabled | |
| S100A1 | NeoMK. MS-1801-S | (DAK-S100A1/1) | Mouse | 1:40 | CC1 standard | 40 min. @ 37° C. | RCC |
| S100A6 | Sigma. S5049 | Clone CACY-100 | Mouse | 1:1000 | Protease 1 | 32 min. @ 37° C. | #2 |
| | | | | | 4 minutes | | |
| S100P | BD. 610307 | Clone 16 | Mouse | 1:200 | CC1 mild | 48 min.@ 37° C. | Urothelial CA |
| Sal-like protein 4 | BiocareM. CM 384C | 6E3 | Mouse | 1:100 | CC1 standard | 40 min. @ 37° C. | Seminoma |
| (SALL4) | | | | | | | |
| SATB2 | SantaC | SATBA4B10 | Mouse | 1:20 | CC1 standard | 44 min. @ 36° C. | Colon ADC |
| | Sc-81376 | | | | | | |
| SOX2 | CellMq | SP76 | Rabbit | 1:100 | CC1 standard | 32 min. @ 36° C. | Tonsil |
| | 371R | | | | | | |
| SOX10 | CellMq | Polyclonal | Rabbit | 1:100 | CC1 52 min. | 32 min. @ 36° C. | Melanoma |
| | 383A | | | | | | |
| Stathmin | 1972-1 | Rabbit | unk | 1:4800 | CC1 standard | 4 min. @ 36° C. | Cervical SQCC |
| SV40 (BK Virus) | EMD. DP02 | Pab416 | Mouse | 1:100 | CC1 standard | 32 min.@ 37° C. | BK virus |
| | | | | | | | (transplanted |
| | | | | | | | kidney) |
| Synaptophysin | BiocareM. PM371 | 27G12 | Mouse | Predilute | CC1 standard | min. @ 37° C. | #4 |
| (Synap) | | | | | | | |
| Syndecan- | Ventana 760-4248 | B-A38 | Mouse | Predilute | CC1 mild | 28 min. @ 37° C. | Tonsil |
| 1(CD138) | | | | | | | |
| TAG-72 (B72.3) | CellMq. 337M-86 | B72.3 | Mouse | 1:100 | CC1 mild | 24 min. @ 37° C. | #2 |
| Terminal | CellMq | Polyclonal | Rabbit | Predilute | CC1 Mild | 32 min. @ 37° C. | Lymphoblastic |
| Deoxynucleotidyl | 338A | | | | | | lymphoma |
| Transferase (TdT) | | | | | | | |
| Thyroglobulin | DAKO. M0781 | DAK-Tg6 | Mouse | 1:200 | None | 32 min32 min. @ | Thyroid |
| (TGB) | | | | | | 37° C. | |
| Thyroid | Ventana 790-4398 | 8G7G3/1 | Mouse | Predilute | CC1 mild | 32 min.@ 37° C. | Lung CA |
| Transcription | | | | | | | |
| Factor 1 (TTF1) | | | | | | | |
| TLE1 | CellMq | 1F5 | Mouse | Predilute | CC2 24 min. | 24 min. @ 36° C. | Synovial sarcoma |
| | 401M | | | | | | |
| TFF1 | Epitomics | EPR3972 | Rabbit | 1:2000 | CC1 mild | 40 min.@ | Colon |
| | E100004 | | | | | 36° C. | |
| | RUO | | | | | | |
| Uroplakin II | BiocareM | BC21 | Mouse | Predilute | CC1 mild | 32 min.@ 37° C. | Urothelial |
| | AVI3051G | | | | | | carcinoma |
| Villin | Ventana 760-4277 | CWWB1 | Mouse | Predilute | CC1 mild | 32 min.@ 37° C. | #2 |
| Vimentin (Vim) | Ventana 790-2917 | V9 | Mouse | Predilute | CC1 mild | 32 min.No heat | #2 |
| Von Hippel- | SantaC | Polyclonal | Rabbit | 1:150 | Protease 1 | 40 min.@ 37° C. | Nomal Kidney |
| Lindau (VHL) | Sc-5575 | | | | 8 min. | | |
| Wilms Tumor | NeoMK. RB-9267P | Polyclonal | Rabbit | 1:200 | CC1 mild | 20 min. @ 42° C. | Renal CA |
| Gene 1 (WT1) | | | | | | | |

EBV and HPV are done by in situ hybridization, which are not included in the above table.

### Example 6: Construction of Tissue Microarray (TMA) Blocks

Tissue microarray (TMA) blocks may be created using standard techniques known to those skilled in the art. For example, TMA blocks were created using the TMA Grand Master (3DHISTECH Kft-PerkinElmer, Waltham, Mass.). The software 2.0.10.3811 was applied in the procedure; a core-size of 2 mm was determined and sample-sites from donor cell blocks were selected. The drilling, coring, implanting and record keeping were automatic. Using this technique, 6 TMA receipt blocks containing 2 2.0 mm punch cores can be created using one donor cell block.

### Example 7: Confirmatory Testing of Select Biomarkers on TMA Blocks Containing Mixed Cell Lines

Immunohistochemical assays for select biomarkers were performed on a group of specific TMA blocks containing the mixed cell lines with the above-identified ratios. These TMA blocks tested included 1) melanoma control block; 2) breast cancer control block; 3) lymphoma control block; 4) germ cell tumor control block; 5) sarcoma control block; 6) malignant small round cell tumor control block; and 7) tumor of unknown primary origin control block.

Tables 6 through 12 summarize the results of each specific TMA control block.

TABLE 6
Summary of a mixture of cell lines and ratios for constructing the melanoma
control TMA block

| | | | | | Mixture of |
|---|---|---|---|---|---|
| | | | | | G361 (40%), |
| | | | | | CRL-1585 |
| Positive | | CRL-1585 cell | A375.52 cell | CRL-5895 cell | (40%), and |
| biomarkers | G361 cell line | line | line | line (lung | CRL-5895 |
| tested | (melanoma) | (melanoma) | (melanoma) | cancer) | (20%) |
| S100 | 2+, W (Low) | 4+, S (High) | 4+, S (High) | Null | High and |
| | | | | | low/null |
| | | | | | expression |
| HMB45 | 4+, S (High) | Null | Null | Null | High and |
| | | | | | low/null |
| | | | | | expression |
| MiTF | 1+, W (Low) | 4+, S (High) | 4+, S (High) | Null | High and |
| | | | | | low/null |
| | | | | | expression |
| Mart-1 | 4+, S (High) | 3+, S (High) | 2+, W (Low) | Null | High and |
| | | | | | low/null |
| | | | | | expression |
| SOX10 | 4+, S (High) | 3+, S (High) | 2+, S | Null | High and |
| | | | | | low/null |
| | | | | | expression |
| SOX2 | 2+, W (Low) | 3+, S (High) | Null | Null | High and |
| | | | | | low/null |
| | | | | | expression |
| MUM1 | Null | 3+, S (High) | 1+, S (Low) | Null | High and |
| | | | | | low/null |
| | | | | | expression |
| S100A6 | Null | 4+, S (High) | 4+, S (High) | Null | High and |
| | | | | | low/null |
| | | | | | expression |
| Vimentin | 4+, S (High) | 4+, S (High) | 4+, S (High) | Null | High and |
| | | | | | low/null |
| | | | | | expression |
| Cytokeratin | Null | Null | Null | 4+, S (High) | High and |
| | | | | | low/null |
| | | | | | expression |
| Cytokeratin 7 | Null | Null | Null | 4+, S (High) | High and |
| | | | | | low/null |
| | | | | | expression |

Table 6 shows the cell lines used to construct the melanoma control block and the relevant expression patterns thereof. The last column demonstrates the mixture of the 3 cell lines used to make up the melanoma control block. Melanoma is a great mimicker for a broad spectrum of both benign and malignant tumors including carcinomas, lymphomas, sarcomas, and germ cell tumors. When working on an undifferentiated tumor, melanoma is nearly always included in the diagnostic consideration. S100, HMB45, MiTF, Mart-1, and SOX10 are a group of the most sensitive and specific biomarkers for melanoma. To render a diagnosis of malignant melanoma, 2-3 of these markers are usually needed. As illustrated in Table 6, none of the three melanoma cell lines demonstrated a high expression (meaning 3+ or 4+, S or I) for the group of aforementioned 5 important diagnostic markers. However, when combining G361 and GRL1585, all melanoma related biomarkers are effectively covered. GRL5895 is a lung cancer cell line to provide an internal control for cytokeratins as negative biomarkers, since carcinoma is frequently included in the differential diagnosis.

A similar strategy has been employed to create other TMA blocks including the control blocks for breast cancer, lymphoma, germ cell tumor, sarcoma, malignant small round cell tumor, and tumor of unknown primary.

TABLE 7
Summary of a mixture of cell lines and ratios for constructing the
breast cancer control TMA block

| | cell lines | | |
|---|---|---|---|
| Biomarkers | HTB-133 (40%) | CRL-2330 (40%) | CCL-253 (20%) |
| HER2 | 1+ and 2+, I | 3+, S (High) | Null |
| ER | 4+, S (High) | Null | Null |
| PR | 3+, I (High) | Null | Null |
| GATA3 | 4+, S (High) | 2+, I | Null |
| GCDPF15 | 3+, S (High) | Null | Null |
| AE1/3 | 4+, S (High) | 4+, S (High) | 4+, S (High) |
| CK7 | 4+, S (High) | 4+, S (High) | 4+, S (High) |
| CK20 | Null | Null | Null |
| MIB-1 (Ki-67) | 4+, S (High) | 4+, S (High) | 4+, S (High) |

HER2 interpretation and scoring system are based on the CAP/ASCO guidelines.

TABLE 8
Summary of a mixture of cell lines and ratios for constructing the
lymphoma control TMA block

| | cell lines | | |
|---|---|---|---|
| Biomarkers | CRL-1582 (40%) | CCL-86 (40%) | CRL-5895 (20%) |
| CD2 | 2+, S | Null | Null |
| CD3 | 4+, S (High) | Null | Null |
| CD5 | 2+, W (Low) | Null | Null |
| CD10 | Null | 2+, I | Null |
| CD20 | Null | 4+, S (High) | Null |
| CD79a | Null | 4+, S (High) | Null |
| Pax5 | Null | 4+, S (High) | Null |
| Bcl-2 | Null | 2+, W(Low) | Null |
| Bcl-6 | Null | 4+, S (High) | Null |
| Tdt | 3+, S (High) | Null | Null |
| CD34 | 1+, S | Null | Null |
| EBV (in situ) | Null | 2+, S | Null |
| CK (AE1/3) | Null | Null | 4+, S (High) |
| CK7 | Null | Null | 4+, S (High) |
| KOC (IMP3) | Null | Null | 4+, S (High) |

TABLE 9
Summary of a mixture of cell lines and ratios for constructing the
germ cell tumor control TMA block

| | cell lines | | | |
|---|---|---|---|---|
| | CRL-2073 | HTB-36 | HB-8065 | CRL-1585 |
| Biomarkers | (25%) | (25%) | (25%) | (25%) |
| SALL4 | 4+, S (High) | 4+, S (High) | Null | Null |
| OCT4 | 4+, S (High) | Null | Null | Null |
| PLAP | 1+, S | 2+, S | Null | Null |
| AFP | 1+, S | Null | 4+, S (High) | Null |
| Glypican 3 | 2+, S | Null | 4+, S (High) | Null |
| CD30 | 3+, S (High) | Null | Null | Null |
| D2-40 | 3+, S (High) | Null | Null | Null |
| SOX2 | Null | Null | Null | 3+, S (High) |
| Nanog | 4+, S (High) | Null | Null | Null |
| S100P | Null | 2+, S | Null | Null |
| CD10 | Null | 4+, S (High) | Null | Null |
| Beta-HCG | Null | 2+, S | Null | Null |
| MOC31 | 3+, S (High) | 2+, S | 4+, S (High) | Null |
| CAM5.2 | 3+, S (High) | 3+, S (High) | 4+, S (High) | Null |
| S100 | Null | Null | Null | 4+, S (High) |
| Vimentin | Null | Null | Null | 4+, S (High) |

The diagnostic biomarkers shown in Table 9 will cover the frequently seen germ cell tumors including seminoma, embryonal carcinoma, yolk sac tumor, choriocarcinoma, and teratoma.

TABLE 10
Summary of a mixture of cell lines and ratios for constructing the sarcoma
control TMA block

| | cell lines | | | | |
|---|---|---|---|---|---|
| | HTB166 | CCL-136 | CRL-2279 | CRL1585 | CRL-1550 |
| Biomarkers | (20% | (20%) | (20%) | (20%) | (20%) |
| Desmin | Null | 4+, S (High) | Null | Null | Null |
| MyoD1 | Null | 4+, S (High) | Null | Null | Null |
| Myogenin | Null | 3+, S (High) | Null | Null | Null |
| SMA | Null | 3+, I (High) | Null | Null | Null |
| CD99 | 4+, S (High) | Null | Null | Null | Null |
| NKX2.2 | 4+, S (High) | Null | Null | Null | Null |
| Fli-1 | 2+, W (Low) | Null | 4+, S (High) | Null | Null |
| ERG | Null | Null | 4+, S (High) | Null | Null |
| S100 | Null | Null | Null | 4+, S (High) | Null |
| Vimentin | 2+, S | 4+, S (High) | Null | 4+, S (High) | Null |
| AE1/3 | Null | Null | Null | Null | 4+, S (High) |
| CK7 | Null | Null | Null | Null | 4+, S (High) |
| CK5/6 | Null | Null | Null | Null | 4+, S (High) |
| CK903 | Null | Null | Null | Null | 4+, S (High) |
| P63 | Null | Null | Null | Null | 4+, S (High) |
| P40 | Null | Null | Null | Null | 4+, S (High) |
| P16 | Null | Null | Null | Null | 4+, S (High) |

TABLE 11
Summary of a mixture of cell lines and ratios for constructing the malignant
small round cell tumor control TMA block

| | cell lines | | | | |
|---|---|---|---|---|---|
| | CCL-136 | HTB166 | CRL-1803 | CRL-1582 | CCL-86 |
| Biomarkers | (20%) | (20%) | (20%) | (20%) | (20%) |
| Desmin | 4+, S (High) | Null | Null | Null | Null |
| MyoD1 | 4+, S (High) | Null | Null | Null | Null |
| Myogenin | 3+, S (High) | Null | Null | Null | Null |
| SMA | 3+, I (High) | Null | Null | Null | Null |
| CD99 | Null | 4+, S (High) | Null | Null | Null |
| NKX2.2 | Null | 4+, S (High) | Null | Null | Null |
| Fli-1 | Null | 2+, W (Low) | Null | Null | Null |
| Synaptophysin | Null | Null | 4+, S (High) | Null | Null |
| Chromogranin | Null | Null | 3+, S (High) | Null | Null |
| CD56 | Null | 1+, S | 4+, S (High) | Null | Null |
| Calcitonin | Null | Null | 3+, S (High) | Null | Null |
| TTF1 | Null | Null | 4+, S (High) | Null | Null |
| NSE | Null | Null | 3+, S (High) | Null | Null |
| AE1/3 | Null | Null | 4+, S (High) | Null | Null |
| CD3 | Null | Null | Null | 4+, S (High) | Null |
| CD20 | Null | Null | Null | Null | 4+, S (High) |
| Tdt | Null | Null | Null | 3+, S (High) | Null |
| EBV (in situ) | Null | Null | Null | Null | 2+, S |
| Vimentin | 4+, S (High) | 2+, S | 1+, S | 4+, S (High) | 4+, S (High) |

The differential diagnosis of small round cell tumors, as shown in Table 11, may include lymphoma/leukemia, small cell carcinoma/neuroendocrine carcinoma, Ewing's sarcoma/PNET, rhabdomyosarcoma, neuroblastoma, leiomyosarcoma, and desmoplastic small round cell tumor. The above biomarkers will cover this group of differential diagnoses.

TABLE 12
Summary of a mixture of cell lines and ratios for constructing the tumor of unknown primary control TMA block

| | cell lines | | | | | | | |
|---|---|---|---|---|---|---|---|---|
| | HTB133 | CCL-253 | CRL-1803 | CRL-2549 | CRL-1932 | CRL-2279 | CRL-1550 | CRL-5946 |
| Biomarkers | (10%) | (10%) | (10%) | (10%) | (10%) | (10%) | (10%) | (10%) |
| CK (AE1/3) | 4+, S (High) | 4+, S (High) | 4+, S (High) | 4+, S (High) | Null | Null | 4+, S (High) | 4+, S (High) |
| EMA | 4+, S (High) | 4+, S (High) | 4+, S (High) | 4+, S (High) | Null | Null | 4+, S (High) | 4+, S (High) |
| CK7 | 4+, S (High) | Null | 4+, S (High) | 4+, S (High) | Null | Null | 4+, S (High) | 4+, S (High) |
| CK20 | Null | 4+, S (High) | Null | Null | Null | Null | Null | ND |
| CK5/6 | Null | Null | Null | Null | ND | ND | 4+, S (High) | Null |
| CK903 | Null | Null | Null | Null | ND | ND | 4+, S (High) | ND |
| CEA | ND | 4+, S (High) | 4+, S (High) | Null | ND | Null | Null | ND |
| MOC31 | ND | 4+, S (High) | ND | 4+, S (High) | ND | Null | ND | ND |
| B72.3 | ND | 4+, S (High) | ND | ND | ND | Null | ND | ND |
| BerEP4 | ND | 4+, S (High) | ND | ND | ND | Null | ND | ND |
| P16 | ND | Null | ND | ND | ND | ND | 4+, S (High) | ND |
| P63 | ND | Null | ND | ND | ND | ND | 4+, S (High) | ND |
| P40 | ND | Null | ND | ND | ND | ND | 4+, S (High) | ND |
| CDX2 | Null | 4+, S (High) | ND | ND | ND | ND | ND | ND |
| SATB2 | Null | 4+, S (High) | ND | ND | ND | ND | ND | ND |
| CDH17 | Null | 4+, S (High) | ND | ND | ND | ND | ND | ND |
| TTF1 | Null | Null | 4+, S (High) | ND | ND | ND | ND | ND |
| ER | 4+, S (High) | Null | ND | ND | ND | ND | Null | ND |
| PR | 3+, I (High) | Null | ND | ND | ND | ND | Null | ND |
| GATA3 | 4+, S (High) | Null | ND | ND | ND | ND | Null | ND |
| GCDFP15 | 3+, S (High) | Null | ND | ND | ND | ND | ND | ND |
| Calcitonin | Null | Null | 3+, S (High) | ND | ND | ND | ND | ND |
| Chromogranin | Null | Null | 3+, S (High) | ND | ND | ND | ND | ND |
| Synaptophysin | Null | Null | 4+, S (High) | ND | ND | ND | ND | ND |
| CD56 | Null | ND | 4+, S (High) | ND | ND | ND | ND | ND |
| Maspin | ND | ND | ND | 4+, S (High) | ND | Null | ND | ND |
| S100P | ND | ND | ND | 3+, S (High) | ND | Null | ND | ND |
| PAX8 | Null | Null | 2+, S | ND | 4+, S (High) | ND | ND | ND |
| PAX2 | Null | Null | ND | ND | 4+, S (High) | ND | ND | ND |
| Vimentin | Null | Null | 1+, S | ND | 4+, S (High) | Null | ND | ND |
| P504S | Null | 3+, S (High) | ND | ND | 4+, I (High) | Null | Null | ND |
| Villin | ND | 4+, S (High) | ND | ND | ND | Null | ND | ND |
| ERG | ND | ND | ND | ND | ND | 4+, S (High) | ND | ND |
| Fli-1 | ND | ND | ND | ND | ND | 4+, S (High) | ND | ND |
| WT-1 | ND | ND | ND | ND | ND | Null | ND | 2+, I |
| Calretinin | ND | ND | ND | ND | ND | Null | Null | 1+, S |
| D2-40 | ND | ND | ND | ND | ND | ND | Null | 2+, I |
| MUC1 | ND | ND | ND | 4+, S (High) | ND | Null | ND | ND |
| MUC2 | ND | 3+, S (High) | ND | ND | ND | Null | ND | ND |
| MUC5AC | ND | ND | ND | 3+, S (High) | ND | Null | ND | ND |
| HPV (in situ) | ND | ND | ND | ND | ND | Null | 4+, S (High) | ND |
| Beta-catenin | ND | 4+, S (High) | ND | ND | ND | Null | ND | ND |
| IMP3 | ND | ND | ND | 3+, I (High) | ND | Null | ND | ND |

A tumor of unknown primary is most frequently encountered in surgical pathology and cytopathology work up, and carcinoma is the most likely diagnosis. The primary site of the undifferentiated carcinoma can originate from the lung, breast, bladder, upper GI, lower GI, pancreatobiliary tract, kidney, uterus, ovary, etc. The biomarkers in Table 12 will cover this broad range of differential diagnoses.

Additional diagnostic biomarkers may be included in the tumor of unknown primary control TMA block. These additional diagnostic biomarkers may include PSA, napsin A, RCC, arginase-1, and HepPar1.

The teachings of all patents, published applications and references cited herein are incorporated by reference in their entirety.

While this invention has been particularly shown and described with reference to example embodiments thereof, it will be understood by those skilled in the art that various changes in form and details may be made therein without departing from the scope of the invention encompassed by the appended claims.

What is claimed is:

1. A method for producing a Tissue Microarray (TMA) Block from a mixture of cultured cell lines for use as an immunohistochemical control block in diagnosis and/or prognosis of one or more particular cancers, the method comprising:
a) identifying a plurality of positive biomarkers useful in diagnosing said one or more particular cancers;
b) optionally measuring the expression of said positive biomarkers in two or more cell lines;
c) selecting:
i) one or more high-positive cell lines that each have a high level of expression of one or more of said positive biomarkers such that the high-positive cell lines collectively provide high level expression of all of said plurality of positive biomarkers; and
one or both of:
ii) one or more low-positive cell lines that each have a low level of expression of one or more of said positive biomarkers; and
iii) one or more null-positive cell lines that each have no expression of one or more of said positive biomarkers;
wherein a single selected cell line may be from both groups i) and ii); i) and iii); ii) and iii); or i), ii), and iii) for different positive biomarkers;
d) mixing cells of said selected cell lines; and
e) producing a TMA block from said mixed cells.

2. The method of claim 1, wherein the low-positive cell lines collectively provide low level expression of the majority of said plurality of positive biomarkers.

3. The method of claim 1, wherein the low-positive cell lines collectively provide low level expression of all of said plurality of positive biomarkers.

4. The method of claim 1, wherein the null-positive cell lines collectively provide no expression of the majority of said positive biomarkers.

5. The method of claim 1, wherein the null-positive cell lines collectively provide no expression of all of said positive biomarkers.

6. The method of claim 1, further comprising:
(a) identifying one or more negative biomarkers useful in diagnosing said one or more particular cancers;
(b) optionally measuring the expression of said negative biomarkers; and
(c) selecting one or more high-negative cell lines that each have a low level or no expression of the plurality of positive biomarkers and high level of expression of one or more of the negative biomarkers;
wherein a single selected cell line may be both a high-negative cell line and a null-positive cell line, or a single selected cell line may be both a high-negative cell line and a low-positive cell line.

7. The method of claim 6, wherein the high-negative cell lines collectively provide high level expression of the majority of said negative biomarkers.

8. The method of claim 6, wherein the high-negative cell lines collectively provide high level expression of all of said negative biomarkers.

9. The method of claim 1, wherein the method, prior to mixing the selected cells, further comprises determining the combination ratio of cells of said selected cell lines required to create a TMA block, wherein said ratio produces a TMA block with about 20-80% of cells having a high level of expression of each of said plurality of positive biomarkers, about 20-80% of cells having a low level of expression of one or more of said positive biomarkers, and about 20-80% of cells having no expression of one or more of said positive biomarkers.

10. The method of claim 6, wherein the method, prior to mixing the selected cells, further comprises determining the combination ratio of cells of said selected cell lines required to create a TMA block, wherein said ratio produces a TMA block with about 20-80% of cells having a high level of expression of each of said plurality of positive biomarkers, about 20-80% of cells having a low level of expression of one or more of said positive biomarkers, and about 20-80% of cells having a high level of expression of each of one or more of said negative biomarkers.

11. A Tissue Microarray (TMA) Block for use as an immunohistochemical control block in diagnosing of one or more particular cancers, wherein the TMA block is selected from the group consisting of:
a) a Melanoma Control Block comprising G361, CRL-1585, and CRL-5895 cells, wherein total cells used are about 40% of said G361 cells, about 40% of said CRL-1585 cells, and about 20% of said CRL-5895 cells;
b) a Breast Cancer Control Block comprising HTB133, CRL-2330, and CCL-253 cells, wherein total cells used are about 40% of said HTB-133 cells, about 40% of said CCL-253 cells, and about 20% of said CRL-2330 cells;
c) a Lymphoma/Hematopoietic Tumor Control Block comprising CRL-1582-Molt4, CCL-86-BLM, and CRL-5895 cells, wherein total cells used are about 40% of said CRL-1582-Molt4 cells, about 40% of said CCL-86-BLM cells, and about 20% of said CRL5895 cells;
d) a Germ Cell Tumor Control Block comprising CRL-2073, HTB-36, HepG2, and CRL-1585 cells, wherein total cells used are about 25% of said CRL-2073 cells, about 25% of said HTB-36 cells, about 25% of said HepG2 cells, and about 25% of said 09-c-CRL-1585 cells;
e) a Malignant Small Round Cell/Blue Cell Tumor Control Block comprising CCL-136, HTB166, TT, CRL-5946, CRL-1582-Molt4, and CCL-86-BLM cells, wherein total cells used are about 20% of said CCL-136 cells, about 20% of said HTB166 cells, about 20% of said TT cells, about 20% of said CRL-5946, about 10% of said CRL-1582-Molt4, and about 10% of said CCL-86-BLM cells;
f) a Sarcoma/Spindle Cell Neoplasm Control Block comprising HTB166, CCL-136, CRL-2279, CRL-1585, and CRL-1550 cells, wherein total cells used are about 20% of said HTB166 cells, about 20% of said CRL-136 cells, about 20% of said CRL-2279 cells, about 20% of said CRL-1585 cells, and about 20% of said CRL-1550 cells; and
g) a Tumor of Unknown Primary Control Block comprising HTB133, NCI-H508, TT, Pan 3.27, CRL-1932, CRL-2279, CRL-1550, and CRL-5946 cells, wherein total cells used are about 15% of said HTB133 cells, about 15% of said NCI-H508 cells, about 20% of said TT cells, about 10% of said Pan3.27 cells, about 10% of said CRL-1932 cells, about 10% of said CRL-2279 cells, about 10% of said CRL-1550 cells, and about 10% of said CRL-5946 cells.

12. A Tissue Microarray (TMA) Block for use as an immunohistochemical control block in diagnosing of one or more particular cancers, wherein the TMA block is selected from the group consisting of:
A) a Melanoma Control Block comprising cells of a plurality of cultured cell lines, wherein said cultured cell lines collectively provide high-level expression and no expression of positive biomarkers S100, HMB45, MiTF, Mart-1, SOX10, SOX2, MUM1, S100A6, and Vimentin; low-level expression of a plurality of biomarkers S100, HMB45, MiTF, Mart-1, SOX10, SOX2, MUM1, S100A6, and Vimentin; and high-level expression and no expression of negative biomarkers Cytokeratin and Cytokeratin 7;
B) a Breast Cancer Control Block comprising cells of a plurality of cultured cell lines, wherein said cultured cell lines collectively provide high level expression, low level expression and no expression of biomarkers HER2, estrogen receptors (ER), progesterone receptors (PR), GATA3, GCDPF15, mammaglobin, TFF1, TFF3, and CK7.
C) a Lymphoma/Hematopoietic Tumor Control Block comprising cells of a plurality of cultured cell lines, wherein said cultured cell lines collectively provide high level expression, low level expression and no expression of biomarkers CD2, CD3, CD5, CD7, CD10, CD20, CD79a, PAX5, Bcl2, Bcl6, EBV, TdT, CD99, CK and CK7;
D) a Germ Cell Tumor Control Block comprising cells of a plurality of cultured cell lines, wherein said cultured cell lines collectively provide high level expression, low level expression and no expression of, biomarkers SALL4, OCT4, PLAP, beta-HCG, alpha-fetoprotein, glypican 3, D2-40, CD30, SOX2, Nanog, S100P, and cytokeratin;
E) a Malignant Small Round Cell/Blue Cell Tumor Control Block comprising cells of a plurality of cultured cell lines, wherein said cultured cell lines collectively provide high level expression, low level expression and no expression of biomarkers desmin, MyoD1, myogenin, smooth muscle actin, CD99, NKX2.2, Fli-1, synaptophysin, chromogranin, CD56, NSE, WT-1, vimentin, TTF1, cytokeratin, CD2, CD3, TdT, CD20, CD79a, and EBV;
F) a Sarcoma/Spindle Cell Neoplasm Control Block comprising cells of a plurality of cultured cell lines, wherein said cultured cell lines collectively provide high level expression, low level expression and no expression of biomarkers desmin, MyoD1, myogenin, smooth muscle actin, CD99, NKX2.2, Fli-1, ERG, vimentin, S100, CK5/6, CK903, p63, p40, p16, and cytokeratin; and
G) a Tumor of Unknown Primary Control Block comprising cells of a plurality of cultured cell lines, wherein said cultured cell lines collectively provide high level expression, low level expression and no expression of biomarkers pan cytokeratin, CK7, CK20, CK5/6, CK903, p63, p40, ER, PR, GATA3, GCDFP15, CDX2, SATB2, cadherin-17, CEA, MOC31, BerEP4, beta-cateinin, B72.3, arginase-1, HepPar1, TTF1, calcitonin, chromogranin, synaptophysin, CD56, MUC1, MUC2, MUCSAC, maspin, S100P, PAX2, PAX8, vimentin, P504S, ERG, Fli-1, p16, WT-1, and HPV.

13. A Universal Tissue Microarray Block for use as an immunohistochemical control block in differential diagnosing of one or more of carcinoma, melanoma, germ cell tumor, sarcoma, and lymphoma comprising G361, CRL-1585, CRL-1582-Molt4, CCL-86-BLM, CRL-2073, HTB-36, HepG2, CCL-136, HTB166, HTB133, CCL-253, NCI-H508, TT, Pan 3.27, CRL-1932, CRL-2279, CRL-1550, and CRL-5946 cells; wherein each cell line comprises about 1 to about 25% of the total cells of said TMA block.

14. A Universal TMA Control Block for use as an immunohistochemical control block in differential diagnosis of one or more of carcinoma, melanoma, germ cell tumor, sarcoma, and lymphoma comprising cells of a plurality of cultured cell lines, wherein said cultured cell lines collectively provide high level expression, low level expression and no expression of biomarkers S100, HMB45, MiTF, Mart-1, SOX2, SOX10, SALL4, OCT4, PLAP, beta-HCG, AFP, glypican 3, CD30, D2-40, HER2, ER, PR, GATA3, GCDFP15, MGB, CK7, CK20, CK5/6, CK903, p40, p63, CDX2, SATB2, cadherin-17, TTF1, napsin A, calcitonin, chromogranin, synaptophysin, CD56, MUC1, MUC2, MUC5AC, calretinin, MOC31, BerEP4, Arginase-1, HepPar-1, PAX2, PAX8, IMP3, maspin, vimentin, p16, WT-1, HPV, desmin, MyoD1, myogenin, SMA, NKX2.2, Fli-1, ERG, CD2, CD3, CD5, CD10, CD20, CD79a, PAX5, TdT, EBV, Ki-67, p53.
